(19) United States
(12) Patent Application Publication
(10) Pub. No.: US 2019/0076631 A1
(43) Pub. Date: Mar. 14, 2019

(54) MICRODEVICES WITH COMPLEX GEOMETRIES

(71) Applicant: Massachusetts Institute of Technology, Cambridge, MA (US)

(72) Inventors: Kevin McHugh, Watertown, MA (US); Ana Jaklenec, Lexington, MA (US); Robert S. Langer, Newton, MA (US)

(21) Appl. No.: 16/130,368

(22) Filed: Sep. 13, 2018

Related U.S. Application Data
(60) Provisional application No. 62/558,172, filed on Sep. 13, 2017.

(51) Int. Cl. A61M 31/00; A61K 9/16; A61M 5/142; A61K 39/00; A61K 9/127; A61B 5/145

(52) U.S. Cl. CPC A61M 31/002; A61K 9/1652; A61B 5/14539; A61K 39/0005; A61K 9/1273; A61M 5/14276

(57) ABSTRACT

Microdevices with complex three-dimensional (3D) internal and external structures are described. The microdevices are made by a method combining micromolding and soft lithography with an aligned sintering process. The microfabrication method, termed StampEd Assembly of polymer Layers (SEAL), generates microdevices with complex geometries and with fully-enclosed internal cavities containing a solid or liquid. The microdevices are useful for biomedical, electromechanical, energy and environmental applications.

### CROSS-REFERENCED TO RELATED APPLICATIONS

This application claims the benefit of and priority to U.S. Provisional Application No. 62/558,172, filed Sep. 13, 2017, which is hereby incorporated herein by reference in its entirety.

### FIELD OF THE INVENTION

The invention is generally directed to microdevices and methods of making and using the microdevices.

### BACKGROUND OF THE INVENTION

Three-dimensional (3D) microstructures have potential use in a wide array of biomedical (tissue engineering and drug delivery), electromechanical (sensors and actuators), energy, and environmental applications. Although a number of extrusion, sintering, and light-based additive manufacturing processes such as 3D printing have been developed to create these devices, each of these methods has advantages and disadvantages that makes it applicable to only a subset of microstructures. Typically, the most appropriate manufacturing technique is selected by considering the size, shape, and composition of the desired microdevice since each technique has limitations in spatial resolution, device geometry, material compatibility, and/or throughput.

For example, stereolithography and fused deposition modeling, which have emerged as leading techniques in the field of custom manufacturing, are subject to a trade-off between resolution and the materials that can be printed. High-resolution stereolithographic 3D printing can produce nanoscale features, but requires photoactive processing additives (some of which have unknown safety profiles in humans) and is not compatible with materials relevant for biomedical applications, such as poly(lactic-co-glycolic acid) (PLGA) and polycaprolactone. These processes also rely on liquid polymerization or cross-linking and may not be compatible with the encapsulation of drugs or other sensitive molecules due to the presence of the liquid pre-polymer solution that could denature or solubilize the cargo.

Heat-based fused deposition modeling, while theoretically compatible with any thermoplastic polymer, lacks the control needed to create microstructures with high resolution. Single-step micromolding processes, such as particle replication in non-wetting templates (PRINT), are attractive for their nanoscale resolution and throughput. However, these approaches are limited to single-layer geometries that can be released from a mold, which makes it difficult to fabricate structures that have internal architecture or a “top-narrowing” 3D shape.

One of the uses for the three-dimensional microparticles with an internal architecture may be for drug and vaccine delivery.

Vaccines typically must be administered more than once to produce maximal response. This usually requires administration of an initial dose of antigen, followed by one or more booster doses at defined times after the initial administration, typically ten to 60 days later. The need for administration of a booster dose clearly limits the practicality of vaccines in much of the world, as well as increases costs and difficulties in areas of poor access to medical care, refrigeration and sterile conditions, as well as in agricultural applications.

Polymeric microparticles have been proposed as vaccine delivery vehicles. They have the ability to enhance targeting of antigen presenting cells (APCs) and have the potential for controlled, sustained release of antigen, thereby potentially eliminating the need for multiple vaccination doses. Further, the polymer matrix can act as a shield from a hostile external environment and has the potential to reduce adverse reactions and abrogate problems caused by the vaccine strain in immunocompromised individuals.

Poly(lactide-co-glycolide) (“PLGA”) microspheres have been developed for single immunization, with and without burst release. Given the biodegradable nature and sustained release properties that PLGA offers, microspheres formulated from PLGA could be useful for the delivery of vaccines. Summarized in Kirby et al., Chapter 13: Formation and Characterization of polylactide-co-galactide PLGA microspheres (2013). PLGA based microparticles are traditionally produced by double emulsion-solvent evaporation, nano-precipitation, cross-flow filtration, salting-out techniques, emulsion-diffusion methods, jet milling, and spray drying. Summarized in Kirby et al., Chapter 13: Formation and Characterization of polylactide-co-galactide PLGA microspheres (2013). PLGA microspheres can also be formulated to incorporate a range of moieties, including drugs and proteins, which can act as adjuvants or targeting. PLGA particles produced by these methods can be lyophilized and stored for later use and delivery.

Hanes et al., Adv. Drug. Del. Rev., 28: 97-119 (1997), report on attempts to make polymeric micro spheres to deliver subunit protein and peptide antigens in their native form in a continuous or pulsatile fashion for periods of weeks to months with reliable and reproducible kinetics, to obviate the need for booster immunizations. Microspheres have potential as carriers for oral vaccine delivery due to their protective effects on encapsulated antigens and their ability to be taken up by the Peyer's patches in the intestine. The potency of these optimal depot formulations for antigen may be enhanced by the co-delivery of vaccine adjuvants, including cytokines, which are either entrapped in the polymer matrix or, alternatively, incorporated into the backbone of the polymer itself and released concomitantly with antigen as the polymer degrades.

As reported by Cleland et al., J. Controlled Rel. 47(2):135-150 (1997), the administration of a subunit vaccine (e.g., gp120) for acquired immunodeficiency syndrome (AIDS) can be facilitated by a single shot vaccine that mimics repeated immunizations. PLGA microspheres were made that provide pulsatile release of gp120. Microspheres were made using a water-in-oil-in-water microencapsulation process with either methylene chloride or ethyl acetate as the polymer solvent. The protein was released under physiological conditions in two discrete phases: an initial burst released over the first day and after several weeks or months, a second burst of protein was released. The second burst of protein was dependent upon the PLGA inherent viscosity and lactide/glycolide ratio (bulk erosion). However, these microspheres are not suitable for encapsulating complex antigens, such as whole, killed, inactivated, or attenuated viruses. In addition, the polymers change upon storage, and require refrigeration to preserve the encapsulated subunit antigens.

These studies demonstrate that it is possible to achieve a vaccine response using injectable microparticles. However, no such product has ever been approved for human or animal use. It is difficult to achieve effective loading of antigen, uniformity of encapsulation and release, and extremely low levels of solvent not affecting antigenicity.

It is estimated that precluding the need for a “cold chain” for vaccine distribution through the development of thermo-stable formulations could save about $200 million annually. It has been difficult to implement these strategies due to a lack of appropriate cryoprotectant methods. A Summarized in Kirby et al., Chapter 13: Formation and Characterization of polylactide-co-galactide PLGA microspheres (2013). Similar information and disclosure on nanovaccines can be found in Gregory et al., Frontiers in Cell and Infect. Microbio. 3:Article 13 (2013). Nandedkar, J. Biosci. 34:995-1003 (2009). Stabilization of proteins included in microspheres is problematic. A number of types of stabilizing excipients have been studied. Summarized in Kim and Pack, BioMEMS and Biomedical Nanotechnology. 1:19-50 (2006). Additionally, the type of polymer used for microsphere fabrication, its degradation rate, acidity of the degradation products, hydrophobicity, etc., can also impact the stability of incorporated proteins.

There remains a need for microdevices for drug delivery, including antigens for use as vaccines, which do not require cryopreservation, are stable at room temperature and humidity, and offer a tight control over release kinetics.

It is therefore an object of the present invention to provide microdevices with complex three-dimensional geometries stably enclosing an agent and with tunable release kinetics.

It is another object of the present invention to provide injectable or ingestible polymeric formulations releasing of the enclosed agent at two or more times.

It is a further object of the present invention to provide injectable or ingestible polymeric formulations, which do not damage and which can stabilize the enclosed agent.

It is a still further object of the present invention to provide methods and materials for making the microdevices and the injectable and ingestible polymeric formulations.

### SUMMARY OF THE INVENTION

Microdevices with complex three-dimensional geometries formed of non-photoactivatable polymers which are micromolded, and methods of their making and using are disclosed. The microdevices may be used in formulations which can be administered once, and provide release at two or more time periods. Formulations are preferably formed of biocompatible, biodegradable polymers for medical applications. Discrete regions encapsulating agent, alone or in combination with other agents, adjuvants, stabilizers, and release modifiers, are present in the formulations. Agent may be present in excipient at the time of administration, or on the surface of the microdevices, for immediate release, and/or incorporated within the microdevices for controlled release. Agents may be stabilized through the use of stabilizing excipients, such as sugars, salts, buffering agents, or combinations thereof. Devices may also be used as sensors and in non-medical applications.

The devices may release the agent at the time of administration, and at subsequent time periods. Preferred times of subsequent release for vaccines is at ten to 45 days after initial release, optionally at ten to 90 day intervals. In a preferred embodiment for immunization against polio, antigen is released at the time of administration, and two, four and six months thereafter. In a preferred embodiment, leakage between bursts of release is not measurable and release occurs over a narrow time frame.

Studies demonstrate that the structure of the microdevices and the composition of the microdevices may be used to provide tunable controlled release formulations. For example, the selection of polymer composition provides for discrete release of antigen, without overlap, with minimal degradation or damage to the encapsulated antigen. Preferred polymers include polylactic acid (“PLA”), polyglycolic acid (“PGA”), and copolymers thereof (“PLGA”), esters of poly(acrylic acid) esters of poly(methacrylic acid), and copolymers thereof.

Microdevices for biomedical applications can be formulated for oral administration, for subcutaneous, intraperitoneal or intramuscular injection via needle or cannula, for topical application to a mucosal region such as intranasal or rectal, or by scarification to the epidermis.

Methods of making microdevices with complex three-dimensional geometries are described. The methods may include a combination of micromolding and soft lithography with an aligned sintering process. The methods generate microdevices of diverse three-dimensional geometries, including solid microdevices, and core-shell devices.

### BRIEF DESCRIPTION OF THE DRAWINGS

FIGS. 1A-1L are diagrams showing master and PDMS mold 30 fabrication for a two-height layer. Micropatterned polydimethylsiloxane (PDMS) molds 30 were generated using the following steps: chemical vapor deposition of silicon dioxide 22 (FIG. 1A) on silicon 20, primary photoresist 24 spin-coating and exposure (FIG. 1B), unprotected oxide etch (FIG. 1C), photoresist removal with oxygen plasma (FIG. 1D), secondary photoresist 26 spin-coating (FIG. 1E), aligned exposure, and development (FIG. 1F) primary deep reactive ion etch, secondary unprotected oxide etch (FIG. 1G), secondary deep reactive ion etch (FIG. 1H), oxygen plasma treatment (FIG. 1I), final oxide removal (FIG. 1J), silicon mold 20 fluorination and PDMS 30 casting (FIG. 1K), and PDMS curing and delamination (FIG. 1L).

FIGS. 2A and 2B are diagrams showing the assembly of 3D microstructures using the StampEd Assembly of polymer Layers (SEAL) process. The diagram in FIG. 2A shows microstructures 48 fabricated by pressing and heating polymer into a patterned PDMS base mold 40 and delaminating these structures onto a substrate 42 to create the first layer 46. A second layer 44 is then formed using a similar molding process against a Teflon surface, which allows the features to remain in the PDMS mold 50 after cooling. The second layer 44 is aligned, placed into contact with the first layer 46, and sintered using a mild heating step. FIG. 2B shows the alignment and sintering equipment, consisting of a mask aligner 100 retrofitted with a Peltier heater 110. A glass slide 52 containing the first layer 56 is suspended upside down from a fixed mask holder using vacuum while the second layer 54, still in the PDMS mold 60, is placed on the wafer chuck (not shown), aligned using the stage rotation and translation knobs (not shown), put into contact and heated until they fuse. This approach was used to create a variety of microstructures, including stars, letters spelling “MiT,” two-layered tables, and three-layered chairs.

FIG. 3 is a bar graph showing the tensile strength (MPa) of SEAL-bonded layers. The adhesion strength between the caps and bases of devices was statistically similar to the tensile strength of the bulk material for PLGA1 and PLGA2, and PLGA4, but slightly lower (p=0.03) for PLGA3, n=5. Error bars indicate standard error of the mean.

FIGS. 4A, 4B and 4C are diagrams showing SEAL-fabricated controlled release microdevices 76. Devices are fabricated by heating and pressing polymer 64 between a patterned PDMS base mold 62 and a Teflon surface 60 (FIG. 4A), transferring these bases 70 to a new substrate 66 and filling them with a model drug 72 of interest (FIG. 4B), then aligning an array of device caps 74 with drug-filled bases 70 and briefly applying a low amount of heat to sinter the two layers (FIG. 4C).

FIGS. 5A and 5B are diagrams showing single injection vaccination and release from SEAL-fabricated PLGA microdevices 82, 84, and 86. FIG. 5A is a diagram of a syringe containing multiple micromolded devices sufficiently small to pass through an 18-gauge needle 90, wherein each of the devices produces a discrete, delayed pulse of antigen release (FIG. 5C) to mimic current bolus vaccination regimens. FIG. 5B is a diagram of an exemplary single microdevice 86 and its dimensions. In vitro pulsatile release of encapsulated Alexa Fluor 680-labeled 10 kD dextran from SEAL-fabricated devices composed of PLGA1 (FIGS. 5D and 5G), PLGA2 (FIGS. 5E and 5H), and PLGA3 (FIGS. 5F and 5I). FIGS. 5D-5F are graphs of the in vitro cumulative release (%) over time (days) of fluorescently labeled dextran at 37° C. with each line indicating an individual device (n=10). FIGS. 5G-5I are graphs of the normalized average cumulative release (%) over time (days) (n=7-10). Note that this yields a broader release curve even though each device exhibits a sharp pulse since the onset of release can differ slightly in each animal. Error bars indicate the standard error of the mean.

FIG. 6 is a bar graph showing fluorescence (fold change) from Alexa Fluor 647-labeled 10 kD dextran in device cores. The graph displays the increase in signal from fluorescent dextran after release from micromolded devices (excitation/emission: 640/720 nm), n=4. Error bars represent standard error of the mean.

FIG. 7 is a line graph showing that the release kinetics (Cumulative Release (%) over Time (days)) are unaltered by storage processes. PLGA1 devices filled with Alexa Fluor 488-labeled 10 kD dextran did not show any significant difference in release kinetics when used immediately after sealing, after freezing at −20° C. or after lyophilization, n=10. Error bars represent standard error of the mean.

FIG. 8 is a bar graph showing inactivated polio vaccine (IPV) stability (Antigenicity Retained (%)) after encapsulation using SEAL. The brief heating process associated with SEAL did not result in any significant change in trivalent IPV D-antigenicity as determined by monoclonal ELISA. Data normalized to filled, unsealed devices containing IPV, n=3-4. Error bars indicate standard error of the mean.

FIGS. 9A and 9B are line graphs showing in vivo release kinetics (Cumulative Release (%) over Time (days)) from mixed device populations. A mixture of 25 PLGA1 devices containing 10 μg of Alexa Fluor 594-labeled 10 kD dextran, 25 PLGA3 devices containing 10 μg of Alexa Fluor 647-labeled 10 kD dextran, and 25 PLGA4 devices containing 10 μg of Alexa Fluor 680-labeled 10 kD dextran were co-injected into SKH1-Elite mice, imaged regularly to observe their respective release kinetics, and compared to a single device of each device type. The in vivo release kinetics of both (FIG. 9A) PLGA1 and (FIG. 9B) PLGA3 were slightly accelerated by the increase in the number of devices or the other populations of polymers compared to a single device, n=5-7. Error bars indicate standard error of the mean.

FIGS. 10A-10C are graphs showing results from ovalbumin vaccination and storage stability. FIG. 10A is a line graph showing longitudinal geometric mean antibody titers (Log₂ (Titer) over Time (weeks)) and FIG. 10B is a graph showing peak antibody titers (Log₂ (Titer)) achieved by mice treated with a single injection of OVA-containing devices or two bolus injections of OVA in solution, n=5. All peak titer groups were significantly different from those of the control group receiving only methyl cellulose (p<0.001), but significance markers are not shown for clarity. All other differences are indicated by * p<0.05 and ** p<0.01. These results show that a single injection of core-shell devices can induce an immune response that is not only non-inferior to two dose-matched boluses, but also non-inferior to two boluses with double the cumulative dose. FIG. 10C is a bar graph showing that core-shell devices stored desiccated for 30 days at 4° C. release the same amount of ELISA-reactive OVA as freshly prepared devices, n=4. Error bars indicate standard error of the geometric mean.

FIGS. 11A-11D are graphs showing environmentally-triggered release and protection of cargo by the pH-responsive devices. FIG. 11A is a line graph showing pH-responsive devices rapidly released (Cumulative Release (%)) a majority of their encapsulated fluorescently labeled dextran within 1 hr in simulated intestinal fluid (SIF), but demonstrated no release in simulated gastric fluid (SGF) even after 7 hr, n=3. FIG. 11B is a bar graph showing FS 30 D devices were also able to protect OVA from SGF for 18 hr, n=5. FIG. 11C is a line graph showing quantification of fluorescence (Normalized Fluorescence (%)) in mice treated with dye in devices showing a substantial increase at 6 hr, n=3. FIG. 11D is a graph showing the localization of fluorescence in the explanted gastrointestinal tracts of mice treated with soluble or encapsulated dye, n=3. FIG. 11D confirms the localization of dye from the devices in the intestines. Error bars indicate standard error of the mean. ** p<0.01, *** p<0.001. Optical images of SEAL-fabricated microdevices composed of EUDRAGIT® FS 30 D before incubation showed the devices remained stable after 7 hr and 18 hr in simulated gastric fluid (SGF, pH 1.2), but begin releasing after 30 min in simulated intestinal fluid (SW, pH 7.5). Representative longitudinal fluorescence imaging (745/800 nm ex/em) of mice receiving Alexa Fluor 750 in solution or encapsulated in FS 30 D devices administered via gavage was conducted. Fluorescence was immediately visible in the stomach of mice treated with soluble dye, but only appeared lower in the body, likely the intestines, after 6 hr in the group receiving devices (images not shown).

FIGS. 12A-12C are diagrams showing a microfluidic device 200 created using the SEAL method. FIG. 12A shows three layers 220, 240, 260 of PLGA were assembled using the SEAL method. The polymeric structure was then covered in polydimethyl sulfone (“PDMS”) 280 (FIG. 12B) and dissolved in dimethylformamide after PDMS cross-linking leaving behind a 3D microfluidic channel 300 (FIG. 12C).

FIGS. 13A-13J are diagrams showing modifications to the SEAL method. FIG. 13A shows the use of an air bladder 400 which may eliminate the need for ensuring rigid platens are parallel. FIG. 13B shows the use of springs 420 to allow top platen 500 to adjust and apply reasonably uniform pressure across entire surface. FIG. 13C shows the use of a roller 440, which applies localized pressure, equally across material. FIG. 13D shows polymer overflow (“scum”) layer may be removed with a skiving knife 460 prior to filling. FIG. 13E shows a vision system combined with a computer controlled laser 480. FIG. 13F shows a punch 600 and die 620 modification. FIG. 13G shows a coining operation eliminating scum layer, which keeps the top of devices planar, with no alignment required for coining operation. FIG. 13H shows another coining operation which eliminates the scum layer and keeps the top of the devices planar. FIG. 13I shows a modified method with the PDMS mold replaced with a silicon mold 700. FIG. 13J shows a simplified process (no PDMS mold, no scum layer) with an option to form spherical microdevices.

### DETAILED DESCRIPTION OF THE INVENTION

### I. Definitions

As used herein, “microdevice” refers to microstructures with diverse or complex three-dimensional geometric shapes. The devices may be solid polymeric devices with no internal cavities. The devices may have one or more internal cavities, such as a hollow core, one or more hollow chambers, or channels. The devices may have diverse core geometries, external shell geometries, or diverse geometries of both the hollow core and the external shell. For example, the core and the shell may have the same geometric shape, such as a cube-shaped core, and a cube-shaped shell. The core and the shell may have different geometric shapes, such as the core may be a hollow cube, while the shell may be star-shaped, or a cone. The devices may be formed by bringing together a “base” device and a cap.

The devices have microscale external dimensions, such as a length, width, height, or diameter, up to one centimeter in at least one dimension, more preferably having a maximum diameter between 1 micrometer (μm) and 1000 μm. As used herein, the “diameter” of a non-spherical device may refer to the largest linear distance between two points on the surface of the device, or between two points of a non-spherical core. When referring to multiple devices or multiple cores, the diameter of the devices or cores typically refers to the average diameter of the devices or cores. Diameter of devices or cores can be measured using a variety of techniques, including, but not limited to, optical or electron microscopy. The diameter of devices can measured with dynamic light scattering. For spherical devices or cores, the “diameter” is used in the art-recognized dimension.

As used herein “base”, or “bases” in a context of a device refers to a base device, which may or may not have a hollow chamber. The hollow chamber, the base, or both, may have diverse geometries.

As used herein, “cap” or “caps” refers to a structure that is used to cap the base or bases. The cap may have any geometric shape, and the geometric shape may be the same as that of the base, or different.

“Additive manufacturing” or “3D printing” as used herein refers to a process of making a three-dimensional solid object of virtually any shape from a digital model. 3D printing is achieved using an additive process, where successive layers of material are laid down in different shapes or thicknesses. In some embodiments, “3D printing” uses an extruded or solvent based polymer-containing ink (e.g., PLGA, poly(L-lactide) (“PLLA”), etc.) that is jetted or extruded through a nozzle and solidified into a desired shape. The shape can be controlled in the x, y and z directions.

“Micromolding,” as used herein, generally refers to processes suitable for manufacturing parts or devices on a microscale, or processes suitable for manufacturing parts or devices having features or tolerances on a microscale. Exemplary techniques include, but are not limited to, lithography.

The term “biocompatible” as used herein refers to one or more materials that are neither themselves toxic to the host (e.g., an animal or human), nor degrade (if the material degrades) at a rate that produces monomeric or oligomeric subunits or other byproducts at toxic concentrations in the host.

The term “biodegradable” as used herein means that the materials degrade or break down into their component subunits.

“Narrow range of release,” as used herein, generally means that the majority of the agent is released over a specific period of time, such as an hour, hours, a day, a week, a month, etc.

“Antigen” or “Vaccine,” as used herein, refers to any molecule or entity that produces a specific immune response in a host organism, such as a mammal. Most T cell antigens are peptides. B cell antigens may be peptides, lipoproteins, sugars, metals or small molecules, especially if conjugated to a protein.

“Immune response,” as used herein, refers to a specific response to an antigen or vaccine that produces immunity to any future exposure in a host, such as a mammal.

“Water soluble” generally refers to something that dissolves or comes apart in aqueous environment.

“Hydrophilic,” as used herein, refers to molecules which have a greater affinity for, and thus solubility in, water as compared to organic solvents. The hydrophilicity of a compound can be quantified by measuring its partition coefficient between water (or a buffered aqueous solution) and a water-immiscible organic solvent, such as octanol, ethyl acetate, methylene chloride, or methyl tert-butyl ether. If after equilibration a greater concentration of the compound is present in the water than in the organic solvent, then the compound is considered hydrophilic.

“Hydrophobic,” as used herein, refers to molecules which have a greater affinity for, and thus solubility in, organic solvents as compared to water. The hydrophobicity of a compound can be quantified by measuring its partition coefficient between water (or a buffered aqueous solution) and a water-immiscible organic solvent, such as octanol, ethyl acetate, methylene chloride, or methyl tert-butyl ether. If after equilibration a greater concentration of the compound is present in the organic solvent than in the water, then the compound is considered hydrophobic.

As used herein, non-photoactivatable means that no structural changes in the material, for example hardening of the material, occurs as a result of cross-linking or polymerization when exposed to light (UV or visible).

### II. Microdevices and Formulations

Polymeric microdevices with complex three-dimensional geometries, and formulations containing microdevices, are disclosed.

A. Polymeric Microdevices

Polymeric microdevices have diverse three-dimensional geometries, and may be free of, or contain one or more internal cavities, such as a hollow core.

The core may contain an agent.

1. Polymers

Polymers are biocompatible and processible under conditions and using reagents that preserve the agent. The microdevices can be made with hydrophilic polymers, hydrophobic polymers, amphiphilic polymers, or mixtures thereof.

Hydrophilic polymers include cellulosic polymers such as starch and polysaccharides; hydrophilic polypeptides; poly(amino acids) such as poly-L-glutamic acid (PGS), gamma-polyglutamic acid, poly-L-aspartic acid, poly-L-serine, or poly-L-lysine; polyalkylene glycols and polyalkylene oxides such as polyethylene glycol (PEG), polypropylene glycol (PPG), and poly(ethylene oxide) (PEO); poly(oxyethylated polyol); poly(olefinic alcohol); polyvinylpyrrolidone); poly(hydroxyalkylmethacrylamide); poly(hydroxyalkylmethacrylate); poly(saccharides); poly(hydroxy acids); poly(vinyl alcohol), and copolymers thereof.

Examples of hydrophobic polymers include polyhydroxyacids such as poly(lactic acid), poly(glycolic acid), and poly(lactic acid-co-glycolic acids); polyhydroxyalkanoates such as poly3-hydroxybutyrate or poly4-hydroxybutyrate; polycaprolactones; poly(orthoesters); polyanhydrides; poly(phosphazenes); poly(lactide-co-caprolactones); polycarbonates such as tyrosine polycarbonates; polyamides (including synthetic and natural polyamides), polypeptides, and poly(amino acids); polyesteramides; polyesters; poly(dioxanones); poly(alkylene alkylates); hydrophobic polyethers; polyurethanes; polyetheresters; polyacetals; polycyanoacrylates; polyacrylates; polymethylmethacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), as well as copolymers thereof. In certain embodiments, the hydrophobic polymer is an aliphatic polyester. In preferred embodiments, the hydrophobic polymer is poly(lactic acid), poly(glycolic acid), or poly(lactic acid-co-glycolic acid).

Biodegradable polymers can include polymers that are insoluble or sparingly soluble in water that are converted chemically or enzymatically in the body into water-soluble materials. Biodegradable polymers can include soluble polymers crosslinked by hydrolyzable cross-linking groups to render the crosslinked polymer insoluble or sparingly soluble in water.

Amphiphilic polymers are polymers containing a hydrophobic polymer block and a hydrophilic polymer block. The hydrophobic polymer block can contain one or more of the hydrophobic polymers above or a derivative or copolymer thereof. The hydrophilic polymer block can contain one or more of the hydrophilic polymers above or a derivative or copolymer thereof.

In particularly preferred embodiments, the biodegradable polymers are polyesters or polyanhydrides such as poly(lactic acid), poly(glycolic acid), and poly(lactic-co-glycolic acid). Polyesters include homopolymers including glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA,” and caprolactone units, such as poly(ε-caprolactone), collectively referred to herein as “PCL;” and copolymers including lactic acid and glycolic acid units, such as various forms of poly(lactic acid-co-glycolic acid) and poly(lactide-co-glycolide) characterized by the ratio of lactic acid:glycolic acid, collectively referred to herein as “PLGA;” and polyacrylates, and derivatives thereof. Exemplary polymers also include copolymers of polyethylene glycol (PEG) and the aforementioned polyesters, such as various forms of PLGA-PEG or PLA-PEG copolymers, collectively referred to herein as “PEGylated polymers.” In certain embodiments, the PEG region can be covalently associated with polymer to yield “PEGylated polymers” by a cleavable linker.

Exemplary thermoplastic polymers include polyamideimide, polyethersulphone, polyetherimide, polyarylate, oolysulphone, polyamide (amorphous), polymethylmethacrylate, polyvinylchloride, acrylonitrile butadiene styrene, polystyrene, polyetheretherketone, polytetrafluoroethylene, polyamide 6,6, polyamide 11, polyphenylene sulphide, polyethylene terephthalate, polyoxymethylene, polypropylene, high density polyethylene, low density polyethylene, polypropylene (PP), polystyrene (PS), polymethylmethacrylate (PMMA), polyvinylchloride (PVC), natural rubber (NR), polydimethyl siloxane (PDMS), polyoxymethylene (POM), polycarbonate (PC), polyethylene terephthalate (PET), polyetheretherketone (PEEK), nylon 6 (PA6), polyamideimide (PAI), polysulphone (PSul), polyphenylene sulphide (PPS), polyethersulphone (PES), polyetherimide (PEI), polytetrafluoroethylene (PTFE), and liquid crystal polymer (LCP).

The polymers may undergo a phase change based on physical or chemical changes of the environment. Exemplary environmental triggers that may change the polymer's physical or chemical characteristics, such as solubility, degradation rate, crosslinking, and rate of erosion, include changes in temperature, changes in pH, and changes in ionic strength.

The polymer can contain one or a mixture of two or more polymers. The polymer may also contain other entities such as stabilizers, surfactants, or lipids.

2. Structure

The microdevices have a complex three-dimensional geometry. The microdevices may be solid, layered and/or include a hollow core. The layers and/or core may contain agent. The microdevices may include one or more microchannels instead of a core. Microdevices with a core are referred to as core-shell devices.

a. Shape and Size

i. Complex 3D Geometry

Microdevices have a complex three-dimensional (3D) geometry, which includes complex geometrical shapes and micron-sized objects. Exemplary geometrical shapes include a cube, cuboid, cone, tetrahedron, square pyramid, hexagonal pyramid, start, cylinder, rectangular prism, triangular prism, pentagonal prism, octahedron, diamond, ellipsoid, and sphere. Exemplary micron-sized objects include microneedles, micron-sized tools, micron-sized decorative objects, such as letters of an alphabet, micron-sized furniture items, such as a micron-sized table, or a micron-sized chair, and any other solid micron-sized object, with or without a hollow core or a channel.

The microdevices may be of any complex 3D geometry. The core of the microdevices, if present, may have any complex 3D geometry. The geometry of the microdevices and cores, if present, may be governed by the end use of the microdevices. For example, if the microdevices are for drug or vaccine delivery, the microdevice shells may be cuboid, cube, spherical, or ellipsoid in shape, and have a cube-, cuboid-, spherical-, or ellipse-shaped core. If the microdevices are for fluid transfer, then the microdevices may be cuboid in shape, and have internal fluid channels with kinks, turns, or a vertically serpentine arrangement.

ii. Size

Micron-sized objects generally have external dimensions, such as a length, width, height, or diameter, each between 1 micrometer (μm) and 1000 μm, 1 micrometer (μm) and 550 μm, 1 micrometer (μm) and 500 μm, 1 micrometer (μm) and 450 μm, 1 micrometer (μm) and 400 μm, between 1 μm and 350 μm, between 1 μm and 300 μm, between 1 μm and 250 μm, between 1 μm and 200 μm, between 1 μm and 150 μm, and between 1 μm and 100 μm. For example, external dimensions for a cuboid-shaped microdevice may be about 250 μm, about 300 μm, or about 400 μm for length, about 250 μm, about 300 μm, or about 400 μm for width, and about 250 μm, about 300 μm, or about 400 μm for height.

If a hollow core is present, the core generally has nanoscale to microscale core dimensions, such as a length, width, height, or diameter, each between 10 nanometers (nm) and 850 μm, between 10 nm and 800 μm, between 10 nm and 750 μm, between 10 nm and 700 μm, between 10 nm and 650 μm, between 10 nm and 600 μm, between 10 nm and 550 μm, between 10 nm and 500 μm, between 10 nm and 450 μm, between 10 nm and 400 μm, between 10 nm and 350 μm, between 10 nm and 300 μm, between 10 nm and 250 μm, between 10 nm and 200 μm, between 10 nm and 150 μm, between 10 nm and 100 μm, between 10 nm and 50 μm, between 10 nm and 10 μm.

Exemplary dimensions for a cube- or cuboid-shaped hollow core include length, width, and height of about 10 μm, about 20 μm, about 30 μm, about 40 μm, about 50 μm, about 60 μm, about 70 μm, about 80 μm, about 90 μm, about 100 μm, about 110 μm, about 120 μm, or about 130 μm, or about 140 μm, about 150 μm, about 200 μm, about 250 μm, or about 300 μm. For example, dimensions for a cuboid-shaped hollow core may be about 100 μm, about 150 μm, about 200 μm, or about 250 μm for length, about 100 μm, about 150 μm, about 200 μm, or about 250 μm for width, and about 100 μm, about 150 μm, about 200 μm, or about 250 μm for height.

b. Microdevice Composition

The different components of the microdevice may be formed of the same polymer composition, different polymers and/or a mix of two or more polymer compositions in one microdevice. The polymer may also contain excipient, metal, ceramic, or other materials, depending on the purpose. For example, in a core-shell device with a base and a cap, the base may be formed form one polymer, while the cap may be formed from another polymer. In one example, the cap may be of the same polymer composition as that used for forming the base but with chemically modified ends. In another example, the cap may be formed of a polymer that differs from the polymer used for forming the base by inclusion of different monomers, or having a different degree of polymerization, or a different co-polymer ratio, or a different blend.

In one example, when the polymer for forming the base is PLGA, and the PLGA polymer for forming the cap has modified ends, such as ester ends, to aid sealing with the base at lower sealing temperature. Maintaining a low sealing temperature minimizes stress on the agent encapsulated in the core. PLGA caps with ester ends also have increased hydrophobicity, which delays the onset of release.

c. Core and Channels

The microdevice core or channel(s), if present, may be empty or contain a solid or liquid agent. The agent is typically loaded into a core during the process of microdevice formation. Agent may be injected into the channel during or after formation of the microdevice.

d. Loading the Core or Channel

The microdevices with hollow cores may contain one or more agents in the void space of the core. The volume of the core's void space varies with the size of the core, with the size of the device, or both. Typically, the void space allows for loading of between pictogram (pg) and milligram (mg) of agent(s). Exemplary loadings include between 10 pg and 1 mg, such as about 100 pg, 1 μg, 2 μg, 3 μg, 4 μg, 5 μg, 10 μg, 100 μg, and 1 mg. Suitable ranges include between about 100 pg and 10 μg, between 1 μg and 5 μg, between 5 μg and 20 μg, between 15 μg and 50 μg, and between 50 μg and 150 μg.

Generally, the devices with cores have a loading capacity between 1 percent weight to weight (% w/w) and 50% w/w, between 1% w/w and 45% w/w, between 1% w/w and 40% w/w, between 1% w/w and 35% w/w, between 1% w/w and 30% w/w, between 1% w/w and 25% w/w, between 1% w/w and 20% w/w, between 1% w/w and 15% w/w, between 1% w/w and 10% w/w, or between 1% w/w and 5% w/w for loading an agent. For example, individual devices may contain about 2% w/w, 4% w/w, 8% w/w, 5% w/w, 13% w/w, 19% w/w, 20% w/w, or 22% w/w loading capacity for loading an agent. An exemplary loading capacity is a loading of approximately 1 μg, 2 μg, 3 μg, 4 μg, 5 μg, 6 μg, 7 μg, 8 μg, 9 μg, or 10 μg of agent in devices with a loading capacity of 2% w/w.

3. Solvents

Solvents must be biocompatible, since some residue will always be present in the polymeric formulations. Representative polymer solvents include organic solvents such as chloroform, dichloromethane, tetrafluoroethylene, and acyl acetate. The agent can be dissolved in aqueous or aqueous miscible solvents such as acetone, ethanol, methanol, isopropyl alcohol, and mixtures thereof.

4. Agents

The microdevices may enclose one or more agents. Typically, the agent is present in the one or more chambers, the core, or the channels of the microdevice. The microdevices may be loaded with one or more agents suitable for biomedical use, for electromechanical use, or agents for energy and environmental use. The agents may be for any commercial, biomedical, electromagnetic, energy, or environmental use.

a. Agents for Biomedical Microdevices

i. Pharmaceutical Agents

The microdevices may contain pharmaceutical agents. Examples of suitable pharmaceutical agents include therapeutic, prophylactic, nutraceutical and diagnostic agents. These include organic compounds, inorganic compounds, proteins, polysaccharides, nucleic acids or small molecules (having a molecular weight of 2000 D or less, more preferably 1000 D or less.

The microdevices may be useful in encapsulating poorly water soluble pharmaceutical compositions. For example, under the Biopharmaceutical Classification System (BCS), drugs can belong to four classes: class I (high permeability, high solubility), class II (high permeability, low solubility), class III (low permeability, high solubility) or class IV (low permeability, low solubility). Suitable active agents also include poorly soluble compounds; such as drugs that are classified as class II or class IV compounds using the BCS. Examples of class II compounds include: acyclovir, nifedipine, danazol, ketoconazole, mefenamic acid, nisoldipine, nicardipine, felodipine, atovaquone, griseofulvin, troglitazone glibenclamide and carbamazepine. Examples of class IV compounds include: chlorothiazide, furosemide, tobramycin, cefuroxmine, and paclitaxel.

ii. Vaccines

Although described with reference to delivery of vaccines, it will be understood that the microdevices may be used to provide release of a variety of therapeutic, prophylactic, nutraceutical, and/or diagnostic agents. Vaccines must include antigen. They may also include immunomodulators such as adjuvants, or agent biasing a response to T cells to induce tolerance to enhance an immune response. In some cases the antigen is used to enhance an immune response to another antigen, such as a tumor antigen expressed in a cancer which may be included in the microdevice or only released in the tissue, where the antigen serves solely as an adjuvant. An example is BCG administered to enhance an anti-cancer response.

### Infectious Agents

Antigens for delivery include killed or attenuated infectious agents such as bacteria such as Clostridia tetani, viruses such as hepatitis, influenza, and polio, and protozoans such as Plasmodium (malaria) and Leishmania, and components thereof.

TABLE 1
Exemplary antigens which can be used in the Microdevices.

| | Age | | | | | | | | |
|---|---|---|---|---|---|---|---|---|---|
| | | 1 | 2 | 4 | 6 | 12 | 15 | 18 | 19-23 |
| Vaccine | Birth | month | months | months | months | months | months | months | months |

| | | | |
|---|---|---|---|
| Hepatitis B¹ | HepB | HepB | HepB |

| | | | | | |
|---|---|---|---|---|---|
| Rotavirus² | RV | RV | RV² | | |
| Diphteria, | DTaP | DTaP | DTaP | See | DTaP |
| Tetanus, | | | | footnote³ | |
| Pertussis³ | | | | | |

| | | | | |
|---|---|---|---|---|
| Heamophilius | Hib | Hib | Hib⁴ | Hib |
| influenza type b⁴ | | | | |
| Pneumococcal⁵ | PCV | PCV | PCV | PCV |

| | | | |
|---|---|---|---|
| Inactivated | IPV | IPV | IPV |
| Poliovirus⁶ | | | |

| | |
|---|---|
| Influenza⁷ | Influenza (Yearly) |

| | | |
|---|---|---|
| Measles, | MMR | See footnote⁸ |
| Mumps, Rubella⁸ | | |
| Varicella⁹ | Varicella | See footnote⁹ |

| | |
|---|---|
| Hepatitis A¹⁰ | HepA (2 doses) |
| Meningococcal¹⁰ | |

Additional vaccines of great interest in third world countries include polio and smallpox. The following uses polio vaccine as an exemplary vaccine for this application.

IPV Vaccine SSI is an inactivated vaccine used for prophylactic vaccination against paralytic poliomyelitis. IPV Vaccine SSI contains inactivated poliovirus type 1, 2 and 3, propagated in Vero cells.

Contents per dose (0.5 ml):

Inactivated poliovirus type 1 (Brünhilde) 40 D-antigen units;

Inactivated poliovirus type 2 (MEF-1) 8 D-antigen units;

Inactivated poliovirus type 3 (Saukett) 32 D-antigen units;

Medium 199 to 0.5 ml.

The vaccine is manufactured without use of serum and trypsin and does not contain preservatives or adjuvants. Antibiotics are not used in the manufacture. IPV Vaccine SSI contains trace amounts of residual formaldehyde. It is manufactured in Denmark by Statens Serum Institut.

IPV Vaccine SSI is a solution for injection distributed in single-dose vials. For primary vaccination a series of three doses of 0.5 ml is administered.

For booster vaccination of previously primary vaccinated persons one dose of 0.5 ml is administered, at the earliest 6 months after the primary vaccination series. Administration of additional booster doses should take place in accordance with national recommendations for polio immunization. The vaccine should be administered intramuscularly or subcutaneously. The vaccine must not be administered intravascular. The age at the first dose should be at least 6 weeks, and the primary vaccination series should include at least three immunizations, with an interval of at least four weeks. Most countries give IPV using the same schedule as DPT vaccine (typically 2 months, 4 months, and 6 months of age).

The immunogenicity and safety of IPV Vaccine SSI has been investigated in several clinical trials, including clinical trials with combined vaccines for pediatric use. Apart from IPV these trials included vaccine antigens against tetanus, diphtheria, pertussis and Haemophilus influenzae type b.

When initiating immunizations at two months of age, completion of a primary vaccination series of three immunizations with at least 1 month interval can be expected to result in seroconversion to all three types of poliovirus one month after the second immunization. When initiating immunizations before two months of age, and at the earliest at 6 weeks of age, seroconversion rates between 89% and 99% have been demonstrated. Therefore, in such a schedule, a booster dose at 9 months of age or in the second year of life should be considered.

IPV Vaccine SSI can be used for revaccination in infants, pre-school aged children and adults primary immunized with IPV or OPV. IPV Vaccine SSI can be used in mixed IPV/OPV schedules, using one to three doses of IPV followed by one to three doses of OPV. It is recommended to administer IPV before the first dose of OPV. In a mixed IPV/OPV schedule, persistence of protective antibodies after primary vaccination has been shown to last at least 20 years. In an IPV only schedule the persistence of protective SSI recommended dose:

D-antigen type 1-40 DU/ml

D-antigen type 2-8 DU/ml

D-antigen type 3-32 DU/ml

### 10× Trivalent IPV:

D-antigen type 1-327 DU/ml

D-antigen type 2-70 DU/ml

D-antigen type 3-279 DU/ml

### Cancer Antigens

Any protein produced in a tumor cell that has an abnormal structure due to mutation can act as a tumor antigen. These may be tumor-specific antigens. Examples of tumor-specific antigens include the abnormal products of ras and p53 genes. In contrast, mutation of other genes unrelated to the tumor formation may lead to synthesis of abnormal proteins which are called tumor-associated antigens.

Proteins that are normally produced in very low quantities but whose production is dramatically increased in tumor cells, trigger an immune response. An example of such a protein is the enzyme tyrosinase, which is required for melanin production. Normally tyrosinase is produced in minute quantities but its levels are very much elevated in melanoma cells.

Oncofetal antigens are another important class of tumor antigens. Examples are alphafetoproteins (AFP) and carcinoembryonic antigen (CEA). These proteins are normally produced in the early stages of embryonic development and disappear by the time the immune system is fully developed. Thus self-tolerance does not develop against these antigens.

Abnormal proteins are also produced by cells infected with oncoviruses, e.g., Epstein Barr Virus (“EBV”) and Human Papillomavirus (“HPV”). Cells infected by these viruses contain latent viral DNA which is transcribed and the resulting protein produces an immune response. In addition to proteins, other substances like cell surface glycolipids and glycoproteins may also have an abnormal structure in tumor cells and could thus be targets of the immune system.

Many tumor antigens have the potential to be effective as tumor vaccines. In addition to alpha fetoprotein (germ cell tumors, hepatocellular carcinoma) and carcinoembryonic antigen (bowel, lung, breast cancers), examples of tumor antigens include CA-125 (ovarian cancer), MUC-1 (breast cancer, epithelial tumor antigen (breast cancer), and melanoma-associated antigen (malignant melanoma).

iii. Anti-Cancer Agents

Representative anti-cancer agents that can be included as agents in the microdevices include, but are not limited to, alkylating agents, antimetabolites, antimitotics, anthracyclines, cytotoxic antibiotics, topoisomerase inhibitors, and combinations thereof. Other suitable anti-cancer agents include angiogenesis inhibitors including antibodies to vascular endothelial growth factor (VEGF) such as bevacizumab (AVASTIN®), other anti-VEGF compounds; thalidomide (THALOMID®) and derivatives thereof such as lenalidomide (REVLIMID®); endostatin; angiostatin; receptor tyrosine kinase (RTK) inhibitors such as sunitinib (SUTENT®); tyrosine kinase inhibitors such as sorafenib (NEXAVAR®), erlotinib (TARCEVA®), pazopanib, axitinib, and lapatinib; transforming growth factor-α or transforming growth factor-β inhibitors, and antibodies to the epidermal growth factor receptor such as panitumumab (VECTIBIX®) and cetuximab (ERBITUX®).

iv. Imaging Agents

The microdevices may contain imaging agents. Examples of suitable imaging agents include radionuclide-labeled small molecules, such as Technetium99 (^{99m}Tc), F-18 fluorodeoxyglucose, fluorinated compounds, superparamagnetic iron oxide (SPIO), gadolinium, europium, diethylene triamine pentacetic acid (DTPA), 1,4,7,10-tetraazacyclododecane-1,4,7,10-tetraacetic acid (DOTA) and their derivatives, gas, and fluorescent tracers. Suitable modalities with respective tracers are known in the art (Baum et al., Theranostics, 2(5)437-447 (2012)).

b. Agents for Electromechanical Microdevices

Microdevices for electromechanical use may contain agents modifying material properties, improving material's electroconductivity following agent's release, or modifying viscosity of materials. The microdevices with agents for electromechanical use may be applied for a pre-determined, timed release of the agents from the microdevices.

c. Agents for Energy and Environmental

Microdevices

Microdevices for energy and environmental applications may enclose agents that act as sensors for environmental changes, such as pH sensors, or as sinks for reacting with, and neutralizing toxins. The microdevices with agents for environmental use may be applied for a pre-determined, timed release of the agents from the microdevices for example, when there is a need for continuous or intermittent monitoring of a sample's pH.

Sensors may also be used in medicine. For example, low pH or elevated enzymes may be indicative of infection, inflammation or cancer. The microdevices can be designed to detect and release preferentially under such conditions, or to release a diagnostic agent that can be used to identify the area of release.

5. Stabilizing Agents/Excipients

The agents to be incorporated into and/or delivered from the microdevices may be in combination with one or more stabilizing excipients. Alternatively, the stabilizing excipients may be included in the polymer shell, instead of the hollow core of the microdevices. In other forms, the stabilizing excipients are present in both the core and the polymer of the microdevices.

The stabilizing excipients increase the structural stability of thermolabile and/or pH sensitive agents. Exemplary stabilizing agents include sugars, sugar alcohols, amino acids, vitamins, anti-oxidants, salts, buffering agents, polysaccharides, oils, and combinations thereof.

Agents that may benefit from stabilization include proteins, peptides, attenuated viruses, and inactivated viruses.

Antigen stability is defined as the maintenance of antigen structure during formation of the vaccine formulation and at body temperature.

Stabilizing excipients may also be added to stabilize antigens. Sugars are a typical group of stabilizing agents for proteins. Examples include simple sugars such as sucrose, fructose, mannitol, glucose, and trehalose as well as more complex sugars. See Alcock et al., Long-term thermostabilization of live poxviral and adenoviral vaccine vectors at supraphysiological temperatures in carbohydrate glass. Science Translational Medicine, 2(19):19-19ra12 (2010).

Stabilization of the antigen can be determined by antigen specific ELISA in vitro and by measuring the immune response (e.g., IgGs) in animals in vivo. Stability is evaluated during each step of the encapsulation and/or manufacturing process, during storage (at 25° C., room temp, high humidity/high temp conditions, under physiological conditions (pH 7.2, 37° C.) and in vivo (animal models).

a. Sugars and Sugar Alcohols

Exemplary sugars and sugar alcohols include trehalose, mannitol, sorbitol, sucrose, fructose, maltose, dextrose, and combinations thereof.

b. Salts

Exemplary salts useful as stabilizing excipients, or stabilizing excipients, include magnesium chloride, calcium chloride, monosodium glutamate, potassium phosphate, and combinations thereof.

c. Buffering Agents

Exemplary buffering agents include Mg(OH)₂, Al(OH)₃, myristic acid, and combinations thereof. When incorporated into microdevices, the buffering agents minimize changes in pH of release medium. Al(OH)₃ is a known adjuvant and can increase immunogenicity.

d. Other Agents

Other agents suitable as stabilizing agents excipients include maltodextrin, methyl cellulose, (hydroxypropyl)methyl cellulose (HPMC), calcium helptagluconate, carboxymethylcellulose (CMC), silk, glycerol, alginate, ectoines, ubiquitin, gelatin, threonine, peptone, glycine, glutamine, serum albumin, and combinations thereof.

The stabilizing excipients may be used in any combination and in any amount effective to stabilize the agent against temperature, storage, humidity, pH, and oxidation insults. For example, stabilizing agents sucrose, monosodium glutamate, magnesium chloride may be used in effective amounts to stabilize the agent. The buffering agent aluminum hydroxide may be included with the stabilizing agents to control changes in environment's pH as the polymer degrades, or as the microdevice passes through a digestive tract.

6. Agents Increasing Rate or Completeness of Release

Gas-generated burst-release systems may allow for instantaneous release of encapsulated antigen. Pore forming agents which are removed by leaching or lyophilization may also be utilized.

B. Formulations

Microdevices of the same, or different polymer composition, and/or agents for delivery, imaging, or other properties may be combined to form one microdevice formulation. Microdevices enclosing the same or different agent(s) may be combined to form one microdevice formulation. Microdevices differing in polymer composition and the enclosed agent(s) may be combined to form one microdevice formulation. The formulations may include polymeric microdevices without agent(s), which may be used to seal or separate areas of the formulation from other areas, and release at different rates.

Formulations typically include microdevices and a pharmaceutically acceptable excipient.

1. Microdevice Composition

Formulations may differ based on the composition of microdevices and enclosed agents.

a. Microdevices of the Same Composition Enclosing Different Agent(s)

Formulations containing microdevices of the same polymeric composition but enclosing different agents may be formulated to provide two or more different agents simultaneously as the polymer degrades. The formulations may be useful for combination therapies, for co-delivery of drugs, with only a single administration.

b. Microdevices of Different Compositions Enclosing the Same Agent(s)

Formulations containing microdevices with different polymeric composition but enclosing the same agent(s) may be formulated for providing two or more burst releases at two of more time points following a single administration. The formulations may be useful for vaccine therapies and the timing of the burst releases may be tuned to mimic the repeat immunization schedule (Table 1) following a single administration.

c. Microdevices of Different Compositions Enclosing Different Agents

Formulations containing microdevices with different polymeric composition and enclosing different agents may be formulated for providing two or more burst releases at two of more time points as polymers of the different compositions degrade, releasing two or more agents. Based on the composition of the microdevices, the formulations may release the two or more agents with each burst release, or release only one type of agent with one release, and another type of agent with the subsequent release, following a single administration.

These formulations may be useful for vaccine therapies, cancer therapies, or therapies for autoimmune diseases.

2. Pharmaceutically Acceptable Excipients

Pharmaceutically acceptable excipients include compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio, in accordance with the guidelines of agencies such as the Food and Drug Administration. Pharmaceutically acceptable excipients include, but are not limited to, diluents, preservatives, binders, lubricants, disintegrators, swelling agents, fillers, stabilizers, and combinations thereof. These include suspending agents such as sterile water, phosphate buffered saline, saline, or a non-aqueous solution such as glycerol.

### III. Methods of Making the Microdevices

It is essential that the methods used to manufacture the device maintain agent stability, both during processing and at body temperature, and that leakage following formation and administration are minimized. Post-formulation sterilization can typically be accomplished through a combination of sterile manufacturing conditions in combination with methods such as gamma irradiation.

A. Emulsion

Microdevices can be made using standard techniques. One technique is emulsification of a polymer solution in an organic solvent with an aqueous solution. Addition of organic phase to a large volume of non-solvent phase forms a spontaneous single emulsion and the resulting solution is stirred continuously for solvent evaporation. Immediate formation of microspheres occurs. After stirring, microspheres are washed and then dried.

B. Three Dimensional Printing

3D printing could increase consistency of microdevices, allowing for more uniform release, as well as provide a means for making more complex devices such as ‘Micro-rods’, having increased carrying capacity, that could eliminate the need for simultaneous release from multiple microdevices, as well as facilitate scale up.

Three-dimensional (3D) printing is a process of making 3D objects from a digital model. 3D printing is an additive process, where successive layers of material are laid down in different shapes. After each layer is added the “ink” is polymerized, typically by photopolymerization, and the process repeated until a 3D object is created. The recent commercial availability and reduced cost makes 3D printing of biomolecules, including vaccines and pharmaceuticals, attractive for distribution of these compounds to developing countries. This would negate the need to ship a finished product into the country. Instead, a 3D printer at the point of care can print out the required biomolecule from a simple computer program, which can come from anywhere in the world.

The 3D printing workflow can be described in three sequential steps: 1) the powder supply system platform is lifted and the fabrication platform is lowered one layer; 2) a roller spreads the polymer powder into a thin layer; 3) a print-head prints a liquid binder that bonds the adjacent powder particles together. Billiet et al., Biomaterials, 33:6020-6041 (2012). Two kinds of 3D printing techniques are mostly adopted for nanobiomaterial fabrication. One is inkjet printing with the typical printers. Marizza et al., Microelectrionic Engin. 111:391-395 (2013). The other is nanoimprint lithography.

Nanoimprint lithography (NIL) is a fast and cost-efficient technique for fabricating nanostructures. The procedure of NIL is to stack multiple layers of such structures on top of each other; that is, a finished double-layer of structures is covered with a spacer-layer which is planarized using the chemical-mechanical polishing so that a second layer can be processed on top. Liu et al., J. Nanomat. August (2013).

Ink-Jet printing has been used to produce monodisperse PLGA particles. Bohmer et al., Colloids and Surfaces A: Physiochem. Eng. Aspects, 289: 96-104 (2006). Briefly, droplets of a PLGA solution are printed with the ink-jet nozzle submerged into an aqueous phase. This method produces microspheres at predictable and controllable sizes. This technique has been used to create Paclitaxel-loaded monodisperse microspheres. Radulecu et al., Digital Fabrication September:18-21. (2005).Variation of this technology has been used to create multilayer monodisperse microspheres. See Kim and Pack, BioMEMS and Biomedical Nanotechnology, 1:19-50 (2006). Utilizing this technology, microcapsule shell thickness can be varied from less than 2 microns to tens of microns while maintaining complete and well-centered core encapsulation for microcapsules near 50 microns in overall diameter.

Drug delivery rates from microparticles have been varied by providing uniform monodisperse microparticles, mixtures of microparticles of varying sizes, and microparticles having different degradable layers. See Kim and Pack, BioMEMS and Biomedical Nanotechnology, 1:19-50 (2006).

Additional information can be found at internet site store.makerbot.com/replicator2 (2013); Tekin et al., Inkjet printing as a deposition and patterning tool for polymers and inorganic particles. Soft Matter, 4:703-713 (2008); Jang et al., Influence of fluid physical properties on ink-jet printability. Langmuir, 25:2629-2635 (2009); Lan, Design and Fabrication of a Modular Multi-Material 3D Printer., M.Sc. Thesis: Massachusetts Institute of Technology, 2013; and at web site imagexpert.com/site-new/pdf/IXjetXpert.pdf. (2013).

C. Micromolding

Park et al., Biomed. Microdevices, 9:223-234 (2007), describes using micromolding to fabricate polymer microstructures having sophisticated designs. Micromolds were filled with polymer microparticles, to produce microstructures composed of multiple materials, having complex geometries, and made using mild processing conditions. These microparticles are typically prepared using an oil-water, double-emulsion system; spray drying methods; supercritical conditioning methods; and milling methods. In a preferred embodiment, micromolds can be prepared by photolithographically creating a female master mold made of photoresist, molding a male master structure out of polydimethylsiloxane (PDMS) from the female master mold and molding a female replicate mold out of PDMS from the male master structure. Polymer microparticles can be micromolded using temperature/press methods and/or from solvent.

Polymeric microparticles of 1 to 30 μm in size were made from PLA, PGA and PLGA using spray drying and emulsion techniques. These polymer microparticles were filled into PDMS micromolds at room temperature and melted or bonded together, for example, by ultrasonically welding microparticles together in the mold while maintaining the voids inherent in their packing structure. Multi-layered microstructures were fabricated to have different compositions of polymers and encapsulated compounds located in different regions of the microstructures. Molds were filled with solid polymer microparticles instead of a polymer melt to copy microstructures with complex geometries and composed of multiple materials using mild processing conditions. Microparticles can flow easily into the cavities of micromolds at room temperature and low pressure, which facilitates making microstructures with high aspect ratios. Moreover, polymer microparticles can encapsulate chemical compounds, such as drugs, and can be filled into molds in sequential layers to accommodate multiple material compositions. After filling the mold, the final microstructures can be created by welding the microparticles within the mold by plastic welding methods, including thermal and ultrasonic welding as well as solvent and gas based welding.

These same techniques can be used to formulate vaccine formulations, once has identified the polymeric materials and conditions required to obtain a narrow time of release at specific time points following administration.

1. StampEd Assembly of Polymer Layers (SEAL)

The microdevices described herein are produced using StampEd Assembly of polymer Layers (SEAL). The SEAL method creates an array of core-shell polymer devices. First, the polymer of choice, PLGA in this example, is melt pressed using a prefabricated silicone mold. The mold with the devices is then transferred to another substrate where it is peeled off, leaving behind an array of polymer base devices. These are then filled with any drug or vaccine using an ink jet piezoelectric nozzle and then dried. Caps are then aligned with the base devices and sealed. The resulting array of core-shell devices are then removed from the base and stored until use.

a. Molds

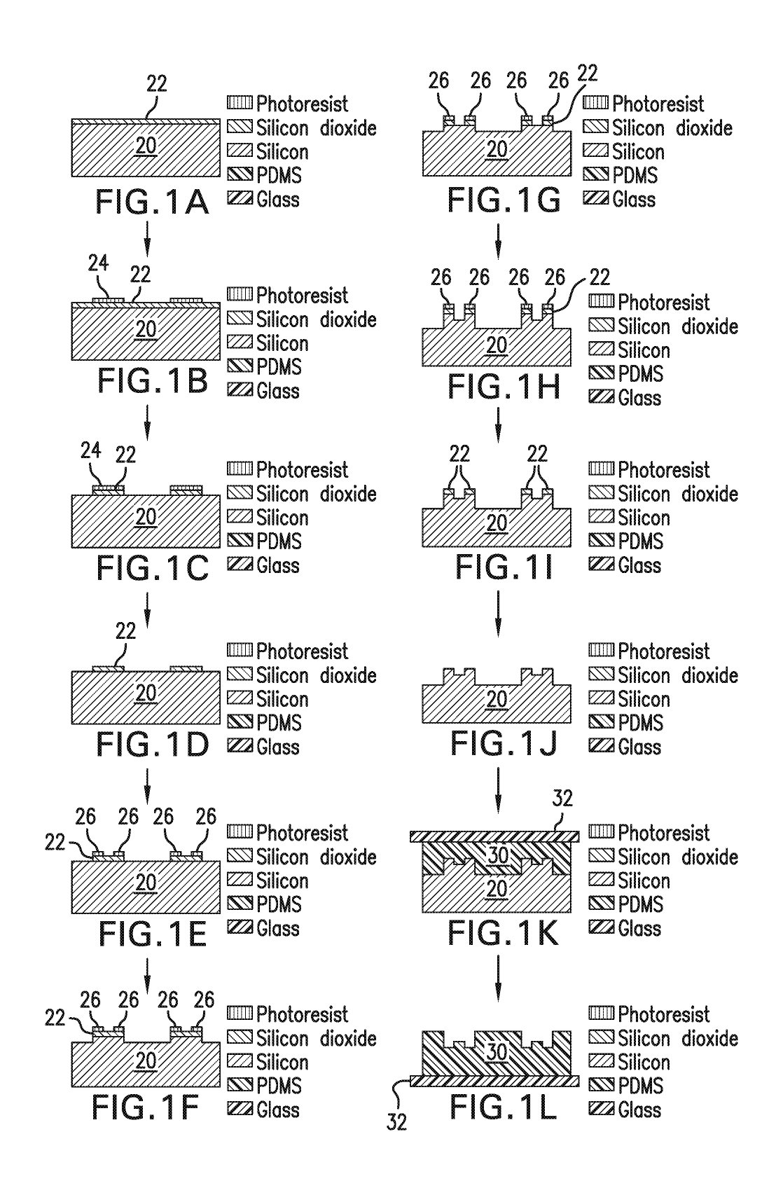

Two or more silicon molds 20 with complementary patterns is etched using standard microfabrication techniques (FIGS. 1A-1J). Polydimethylsiloxane (PDMS) is then cured on the surface of each silicon wafer to produce inverse elastomeric molds 30 (FIGS. 1K-1L). A polymer is then heated and pressed into the PDMS molds to produce laminar microstructure components of interest.

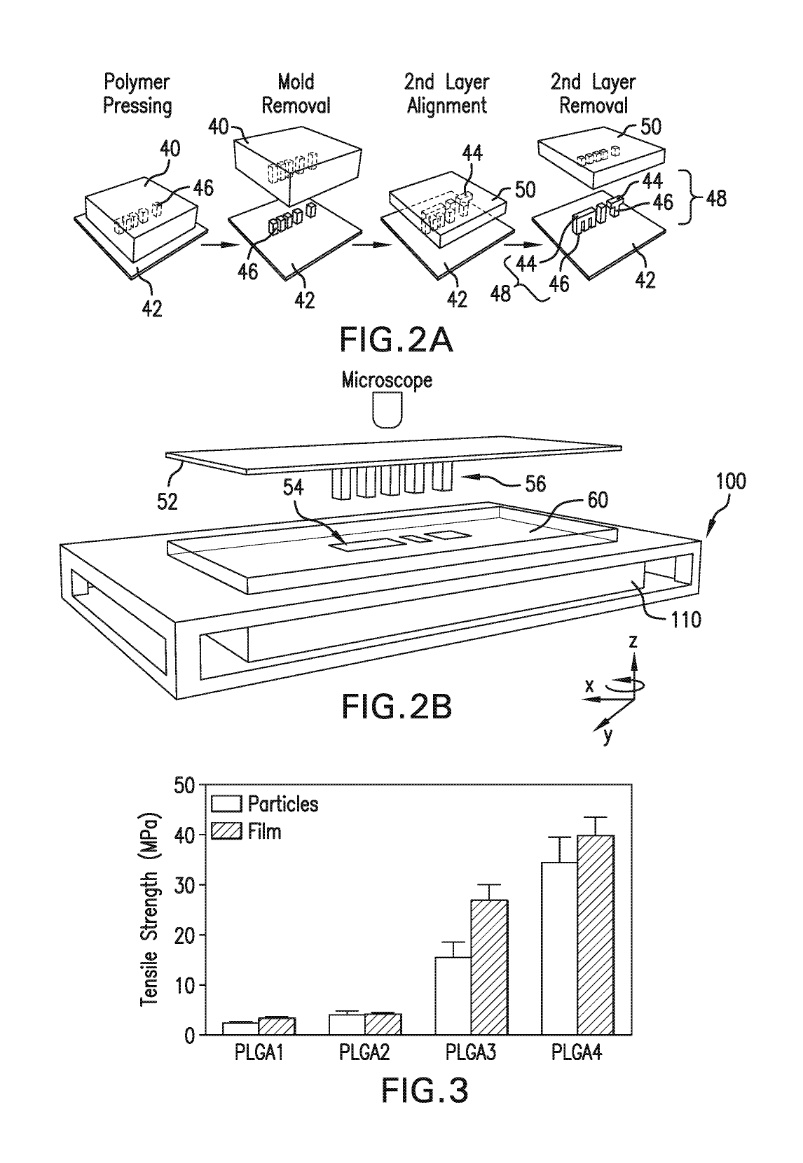

The first layer (e.g., first layer 46) is then delaminated onto a separate surface, such as glass, e.g., substrate 42, using heat-assisted microtransfer molding. Subsequent layers (e.g., second layer 44) of the final structure are then assembled using a layer-by-layer sintering process under microscopic alignment to produce a large array of microstructures (FIGS. 2A and 2B). This process draws on elements from existing technology, including laminated object manufacturing (14), microfabrication-based surface patterning (15), and thermal bonding of PLGA (16), to create discrete polymeric microdevices with well-defined geometry.

b. Layer-by-Layer Alignment and Sintering

To ensure high-fidelity microdevice fabrication, a technique to align layers during sintering with high precision was developed. This approach used a photomask aligner (MA4, Karl Suss, Sunnyvale, Calif.) retrofitted with a Peltier heater 110, temperature controller, relay, and voltage source to enable simultaneous alignment and thermal bonding (FIG. 2B). The mask holder vacuum was applied to hold a glass slide containing the first microstructure layer 56 facing down while the next layer 54, still in the PDMS mold, was held on the wafer chuck. After optically aligning adjacent features using the mask aligner's microscope and alignment knobs, the layers were brought into contact and heated to just above the polymer's glass transition temperature (Table 2) for up to 3 min. The sealing process was continuously monitored during this time by observing the disappearance of light diffraction patterns

As two layers came into contact, the small air gap between them produced diffraction that resolved when the heated polymer flowed to close the gap. After cooling samples to room temperature, the PDMS micromold containing the second layer was peeled off to yield a multi-layered microstructure. Individual microdevices were then removed from the glass slide.

c. Filling and Capping

The micromolded device bases 70 were filled prior to sealing using a BioJet Ultra ink jet piezoelectric nozzle that can rapidly dispense picoliter volumes of model drug (e.g., drug 72) into a device core.

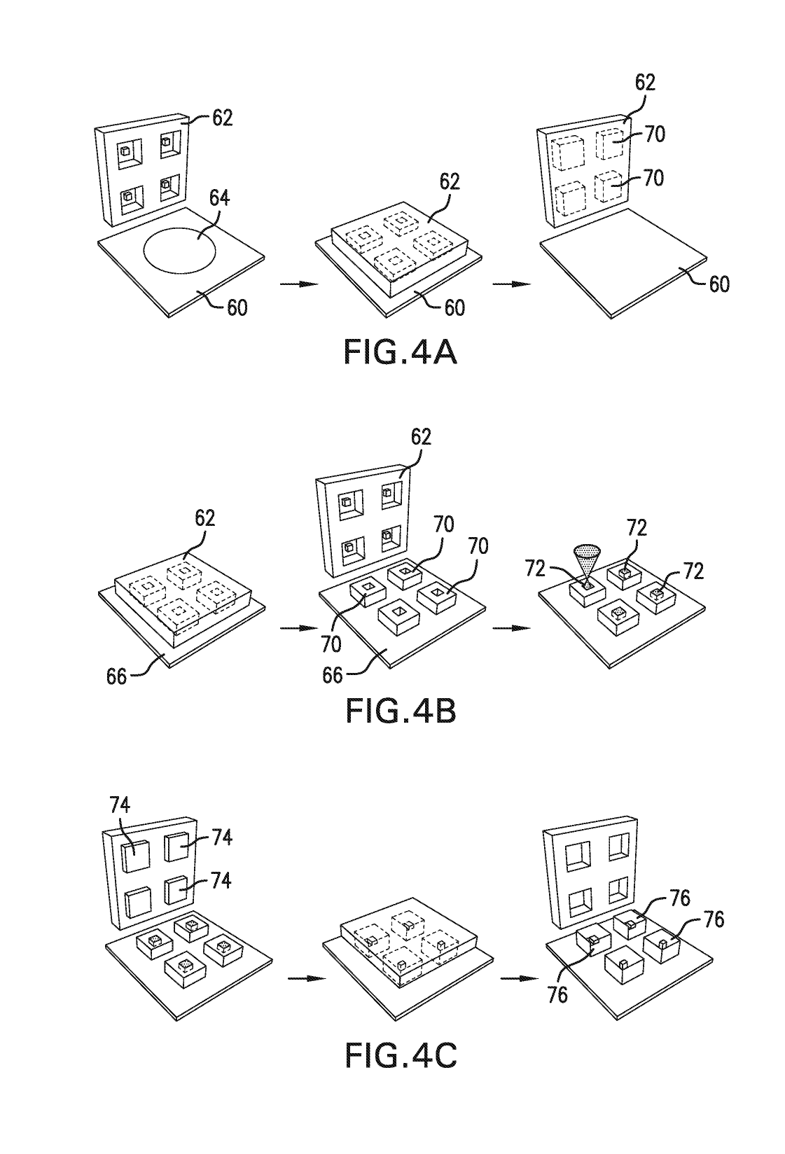

To seal the filled devices, a cap mold (e.g., cap 74) was aligned, sealed with the base 70, and delaminated. The resulting array of core-shell devices 76 was then removed from the base and stored until use (see FIGS. 4B and 4C).

d. Methods of Removing Scrum

In some cases, the polymer used to fill the micromolds forms a “scrum” at the top which should be removed before capping.

The examples demonstrate various ways in which the scrum can be removed.

### IV. Methods of Using the Microdevices

The microdevices and formulations may be adapted for diverse uses, such as biomedical use, electromechanical use, or energy and environmental use. The biomedical use is presented as a non-limiting example.

A. Biomedical Use

1. Vaccination

The formulations containing microdevices may be administered to an individual in need of vaccination. These are administered as a dosage formulation including an effective amount of one or more antigens released in a schedule that elicits a protective effect against the source of the antigen, or to induce tolerance, for example, to a food, insect, toxin, self-antigen, or component thereof. Following a single administration, the formulations provide an immune protection similar to that achieved currently only with repeat booster vaccinations. Therefore, the formulations may obviate the need for repeat immunizations.

Microdevices can be administered by injection, preferably subcutaneously, intraperitoneally, or intramuscularly, for example, under the skin of the back of the upper arm, or to a mucosal surface (orally, intranasally, vaginally, rectally, via the pulmonary route, or other orifice), although injection is preferred if release is to occur over a prolonged period of time of more than a few days.

The dosage form is designed to release a bolus of antigen at the time of administration. This may be achieved by administering a solution or dispersion of antigen in combination with the vaccine formulation providing multiple releases at subsequent times, or the device may be formulated to provide an initial bolus as well as subsequent release(s).

B. Controlled Release

The microdevices and formulations have been developed for controlled release of agents. Particularly, the injectable or ingestible polymeric formulations release the enclosed agent at two or more times, within a short period of time, and in the absence of agent leakage from the microdevices between releases.

The controlled release can be achieved by incorporating microparticles of different polymeric compositions in one formulation. The controlled release can also be achieved from the geometric design of the microdevices. For example, in the core-shell devices, the agent is enclosed in a hollow core, where it is stable and protected from the external environment. It is also prevented from leaking from the microdevice until the microdevice shell is degraded. Once the shell is degraded, the release is rapid (e.g., for PLGA core-shell devices, complete release of the agent is achieved within hours to two days in vitro, and within hours to six days in vivo). The rapid release may be characterized by releasing substantially all of the enclosed agent within a short period of time, such as within an hour, hours, a day, or a week. Unlike solvent evaporation microencapsulation, where the agent is encapsulated with the polymer into a microsphere, the formulations provide de-coupling of device loading and release kinetics.

The formulations also display no measurable leakage of the agent between bursts of release. No measurable leakage may be characterized by less than 10%, 9%, 8%, 7%, 6%, 5%, 4%, 3%, 2%, 1%, or less than 0.5% of the total agent released prior to the burst release. No measurable leakage may also be characterized by undetectable amounts of agent released prior to the burst release.

The formulations display rapid release after material-dependent delay. The delay in agent release is dependent on the shell thickness in the core-shell device, on shell polymer composition, on the geometry of the core-shell device, and other factors.

By combining microdevices of various polymer compositions, core-shell geometry, or particle size, the pulsatile release of the agent(s) can be tailored to a desired controlled release regimen.

C. Adjuvant Effect of Microdevices

The microdevices and formulations may have an adjuvant effect in the absence of a conventional immunologic adjuvant added to the microdevices or formulations. The adjuvant effect may be attributed to the size, shape, and composition of the microdevices themselves.

D. Dose Sparing

As presented in the Examples, the microdevices in the formulation offer stability and protection to the agent to be delivered. The microdevices also provide a tight control of agent release, preventing any agent leakage from the devices in between bursts of release. These effects, combined with the adjuvant effect of microdevices, provides a dose sparing for the effective amount of agent delivered to a subject.

These experiments demonstrate that one injection of core-shell devices is sufficient to achieve the same response to the agent as that achieved after repeat injections of the agent, and with higher doses of the agent. Therefore, the microdevices and formulations reduce the effective dose of the agent required to achieve the same result. The reduction in the effective dose may be 1.5 fold, 2 fold, 2.5 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold, 10 fold, or more. In Example 5 below, the formulations achieve at least a two-fold dose-sparing.

### V. Kits

The microdevices and formulations described above as well as other materials can be packaged together in any suitable combination as a kit useful for performing, or aiding in the performance of, the disclosed method. It is useful if the kit components in a given kit are designed and adapted for use together in the disclosed method. For example, disclosed are kits with one or more dosages packed for injection into a subject, and may include a pre-measured dosage of a formulation in a sterile needle, ampule, tube, container, or other suitable vessel. The kits may include instructions for dosages and dosing regimens.

The present invention will be further understood by reference to the following non-limiting examples.

### EXAMPLES

The Examples show a bottom-up, high-resolution microstructure fabrication technique to create microdevices with complex geometries using a variety of commercially relevant materials, including lactide/glycolide copolymers, the most widely used biodegradable polymers for human applications. This approach, termed the StampEd Assembly of polymer Layers (SEAL), combines the technology used for computer chip manufacturing with soft lithography and an aligned sintering process to produce small (≤400 μm) polymeric structures (FIG. 1).

### Example 1. High-Fidelity Microdevice Base Fabrication

Materials and Methods

Micromold Fabrication

Silicon wafers 20 were patterned with microscale features to create master molds and then replicated in polydimethylsiloxane (PDMS) 30 using soft lithography (FIGS. 1A-1L). Photomasks with patterns corresponding to each layer in the StampEd Assembly of polymer Layers (SEAL) process were created using Layout Editor (Juspertor, Unterhaching, Germany) and made in-house or by Front Range Photomask (Palmer Lake, Colo.). A 3 μm-thick silicon dioxide layer 22 was then deposited on a 150 mm silicon wafer 20 using plasma-enhanced chemical vapor deposition with a recipe of 50 sccm SiH₄ and 800 sccm N₂O for 56.4 sec at 255 W, 400° C., and 2.7 Torr. This wafer was then spin-coated with AZ 4260 photoresist 24 (MicroChemicals, Ulm, Germany) at 1,000 RPM for 60 sec, baked at 95° C. for 1 hr, and then exposed to ultraviolet light through a photomask for 20 sec using an EV620 mask aligner (Electronic Visions, Rockledge, Fla.). Next, exposed photoresist was removed by developing the wafer in MIF405 (MicroChemicals) for 3 min. An oxide etch was then performed using 90 sccm of CF₄, 30 sccm of CHF₃, and 120 sccm of He for 9 min at 850 W and 2.8 Torr to remove the oxide. For layers with multiple desired heights such as the microdevice bases, a second round of photoresist 26 deposition, aligned exposure, development, and etching was performed. The wafer was then etched using a Haber-Bosch process in a Pegasus deep reactive ion etcher (Surface Technology System, Newport, Wales) using an etch recipe of 450 sccm SF₆ and 45 sccm O₂ for 7 sec at 2800 W and passivation recipe of 200 sccm C₄F₈ for 4.5 sec at 2000 W to etch down exposed silicon to the desired height for each layer. After etching, the wafer was cleaned by acetone, isopropanol, and oxygen plasma at 1000 W, 0.9 mTorr O₂ for 1 hr. The Pegasus was then used to deposit approximately 300 nm of C₄F₈ (200 sccm of C₄F₈ for 1 min at 2000 W, 25 mTorr). This polymer film acts like Teflon and limits adhesion to PDMS in subsequent steps. PDMS base and curing agent were mixed in a 7:1 ratio (Sylgard 184, Dow Corning, Midland, Mich.), poured onto the silicon master mold, and degassed under vacuum for 1 hr. A thin PDMS mold 30 was then produced by attaching two cover slips to the end of a glass slide 32 as spacers and pushing down against the silicon mold while curing at 75° C. for 2 hr.

Polymer Micromolding and Transfer Using Soft Lithography

Poly(lactic-co-glycolic acid) (PLGA) films were created by solvent casting 20-60% w/v PLGA in acetone on Teflon-coated glass and passing them under a doctor blade between 300 and 800 μm in height depending on the polymer. These films were then further dried overnight on a 60° C. hot plate to yield rigid films 50-100 μm thick. A small piece of this PLGA film was then placed between the PDMS mold and a Teflon film and compressed under a spring-loaded clamp in a 120° C. oven under high vacuum for 2-24 hr depending on the polymer. After cooling, the clamp and Teflon film were removed and separated yielding PLGA entrapped in the patterned depressions of the PDMS mold. In some cases, a thin film of PLGA on the order of a few hundred nanometers was still present between the microstructures, but did not affect layer-by-layer assembly. To transfer the bottom layer of each structure (such as the microdevice base) to a different substrate, the PDMS mold containing polymer was clamped against a glass slide and heated at 120° C. for 5 min before being cooled with dry ice and separated. These structures could then be separated from the glass slide using forceps or a razor blade.

Layer-by-Layer Alignment and Sintering

Multi-layer polymer structures were precisely aligned and sintered using the method described in the main text. Briefly, a photomask aligner was used to rotate and translate adjacent polymer layers until they were visibly aligned. Layers were then brought into contact and heated until they fused and sealed. Rarely, a device exhibited immediate release after in vivo injection, likely due to handling damage or inadequate manual sealing, and was excluded from the data set. The current manual process relies on visual inspection of diffraction patterns, but could be automated using an optical sensor or interferometer to capture the resolution of diffraction patterns upon sintering. The other challenges associated with the heat-assisted microtransfer molding step in SEAL are the possibility that the polymer does not release from the mold or that a nanoscale film forms that connects otherwise discrete features. However, these challenges can be readily overcome by silanizing the PDMS molds with tricholoro(1H,1H,2H,2Hperfluorooctyl) silane to promote second-layer delamination, compressing the mold with sufficient force to eliminate the connective film, or by replacing PDMS with a fluorinated elastomer as used in the Particle Replication in Non-wetting Templates (PRINT) method. There is likely some limit to the geometries that can be obtained based on the mechanical strength of the material being molded.

Device and Polymer Characterization

Molecular weights were determined using a Viscotek TDA 305 (Malvern Instruments, Malvern, United Kingdom) using tetrahydrofuran as the solvent against poly(methyl methacrylate) standards. Glass transition temperature was determined by running samples on a Perkin Elmer Diamond Differential Scanning calorimeter. Optical images of microstructures were stitched together from multiple images to enable a better depth of focus. The interfaces between images are denoted by thin red lines. High resolution images of devices were collected using a scanning electron microscope (SEM). First, samples were coated with a thin layer of Au/Pd using a Hummer 6.2 Sputtering System (Anatech, Battle Creek, Mich.) and then imaged using a JSM-5600LV SEM (JEOL, Tokyo, Japan) with an acceleration voltage of 5 kV. The brightness and contrast of these images has been modified post-collection for consistency and optimal electronic and print viewing. Mechanical testing was performed using a Dynamic Mechanical Analyzer Q800 (TA Instruments) equipped with a tension clamp. Samples were mounted and a force ramp of 1 N/min was applied. Each sample type was replicated 5 times.

Results

Two or more silicon molds with complementary patterns were etched using standard microfabrication techniques (FIGS. 1A-1J). Polydimethylsiloxane (PDMS) was then cured on the surface of each silicon wafer to produce inverse elastomeric molds (FIGS. 1K-1L). A polymer was then heated and pressed into the PDMS molds to produce the laminar microstructure components of interest. The first layer was then delaminated onto a separate surface, such as glass, using heat-assisted microtransfer molding. Subsequent layers of the final structure were then assembled using a layer-by-layer sintering process under microscopic alignment to produce a large array of microstructures (FIGS. 2A and 2B). This process draws on elements from existing technology, including laminated object manufacturing, microfabrication-based surface patterning, and thermal bonding of PLGA, to create discrete polymeric microdevices with well-defined geometry.

To ensure high-fidelity microdevice fabrication, a technique to align layers during sintering with high precision was developed. This approach used a photomask aligner (MA4, Karl Suss, Sunnyvale, Calif.) retrofitted with a Peltier heater, temperature controller, relay, and voltage source to enable simultaneous alignment and thermal bonding (FIG. 2B). The mask holder vacuum was applied to hold a glass slide containing the first microstructure layer facing down while the next layer, still in the PDMS mold, was held on the wafer chuck. After optically aligning adjacent features using the mask aligner's microscope and alignment knobs, the layers were brought into contact and heated to just above the polymer's glass transition temperature (Table 2) for up to 3 min. The sealing process was continuously monitored during this time by observing the disappearance of light diffraction patterns. As two layers came into contact, the small air gap between them produced diffraction that resolved when the heated polymer flowed to close the gap.

After cooling samples to room temperature, the PDMS micromold containing the second layer was peeled off to yield a multi-layered microstructure. Individual microdevices were then removed from the glass slide. This process was used to create large arrays of microstructures, including a 3D star, two-layered letters spelling out “MiT,” a two-layer table with high-aspect-ratio supports (FIGS. 2A and 2B), and a three-layer chair. Mechanical characterization of the PLGA microstructures indicated that the adhesion strength of adjacent layers was of similar magnitude as that of the corresponding bulk material (FIG. 3).

### Example 2. Filling Microdevices and Varying Device Loading

Materials and Methods

The microdevice bases were prepared as described in Example 1.

Device Filling

A BioJet Ultra picoliter dispensing apparatus was used to fill compounds into the device core. A 50 mg/ml solution of Alexa Fluor 488- or 680-labeled 10 kD dextran (Life Technologies, Carlsbad, Calif.) was used as a model drug for ease of tracking. Microdevice cores were filled with solution using multiple ten-drop cycles of 100-150 pl drops. The volume filled during each cycle could exceed the volume of the device core due to rapid evaporation and convex meniscus formation.

Histology

SKH1-Elite mice were injected subcutaneously with PLGA3 devices containing 400 μg of Alexa Fluor 680-labeled 10 kD dextran to help identify the implant location after degradation. After 2, 4, or 8 weeks mice were euthanized and their skin and adjacent sub-dermal tissue was harvested, fixed in formalin-free fixative (Sigma Aldrich) for 24 hr, and transferred to 70% ethanol. Tissue was then embedded in paraffin, cut into 5 μm-thick tissue sections, stained with hematoxylin & eosin or Masson's trichrome stains, and imaged using an Aperio AT2 Slide Scanner (Leica Biosystems, Buffalo Grove, Ill.).

Results

SEAL enabled the fabrication of complex 3D microstructures that are otherwise challenging to generate, especially using materials such as PLGA. SEAL created a PLGA microdevice platform to deliver timed pulses of antigens for essentially any drug or a vaccine in a single injection. To create these microdevices, fillable bases were molded using thermoplastic polymers and transferred to a glass slide to expose the empty device core (FIG. 4A). Device cores were filled with a model drug solution using a BioJet Ultra picoliter dispensing apparatus (BioDot, Irving, Calif.) (FIG. 4B), aligned with capping polymer lids, pressed together, and briefly heated to seal the devices (FIG. 4A). Scanning electron microscopy mages of these devices after each stage of the fabrication process were taken. Micromolded device bases with a 100×100 μm core and 400×400×200 μm external dimension were filled prior to sealing using a BioJet Ultra ink jet piezoelectric nozzle that can rapidly dispense picoliter volumes of model drug into the device core. Filling was at the default of 4 drops/sec for the purposes of visualization, but the equipment is capable of achieving rates up to 50 drops/sec.

These core-shell devices are useful for biomedical applications because they are sufficiently small to be injected, can be created using fully biodegradable materials, and induce only a minimal foreign body reaction. Hematoxylin & eosin and Masson's trichrome staining of the tissue surrounding a PLGA3 microdevice 2, 4, and 8 weeks after subcutaneous injection showed only a minor foreign body reaction around the PLGA3 device with relatively few macrophages present at the tissue/biomaterial interface, thin fibrous capsule formation, and little evidence of device injection by 8 weeks. The device also appears to largely retain its shape prior to release.

Each device weighs approximately 60 μg and has a theoretical loading of about 2%; however, much higher loading can be achieved by varying the wall thickness to yield larger cores. PLGA1 devices with a variety of internal core and external footprint dimensions resulting in theoretical loading of 2%, 4%, 5%, 8%, 13%, 19%, 20%, and 22% were formed (images not shown).

### Example 3. Timed Pulse of Agent Release from the Microdevices

Materials and Methods

The microdevices were prepared as described in Examples 1 and 2.

In Vitro Release Kinetics

Sealed devices with an outer footprint of 400×400×300 μm and 100×100×100 μm core were filled with 300 ng of Alexa Fluor 488-labeled 10 kD dextran. Each device was then placed into 300 μl of phosphate-buffered saline (PBS) in a lo-bind microcentrifuge tube (Eppendorf, Hamburg, Germany) and incubated on a shaker at 37° C. Release was then measured every 1-4 days depending on device composition by analyzing the supernatant fluorescence at 475/520 nm in technical duplicate with a Tecan Infinite M200 spectrophotometer (Männedorf, Switzerland). Results were quantified using a standard curve and normalized to total cumulative release (n=10). At each time point, supernatant was replaced with 300 μl of fresh PBS. The timing of release is reported as the day at which more than half of the total payload has been released.

In Vivo Release Kinetics

The same types of devices studied in vitro were also used to study in vivo kinetics in female SKH1-Elite mice (SKH1-Hr^{hr}, Charles River Laboratories, Wilmington, Mass.) a hairless, but immunocompetent strain. All procedures were approved prior to beginning in vivo experiments by the MIT Committee on Animal Care. Ten days prior to injection, mice were switched to an alfalfa-free purified rodent diet (Harlan Laboratories, Madison, Wis.) to reduce intestinal auto-fluorescence. Devices were sterilized prior to surgery using a 20 μl drop of 70% ethanol which dried in approximately 1 min. On the day of injection, mice were anesthetized using continuous inhalation of 3% isoflurane and had their injection site sterilized with ethanol.

Devices filled with Alexa Fluor 680-labeled 10 kD dextran were then tip-loaded into a 18 Monoject filter needle (Covidien, Dublin, Ireland) in approximately 100 μl of 15 mg/ml of 4,000 cP methyl cellulose (Sigma Aldrich) used as a viscosity enhancer and injected subcutaneously into the rear flank of the animal.

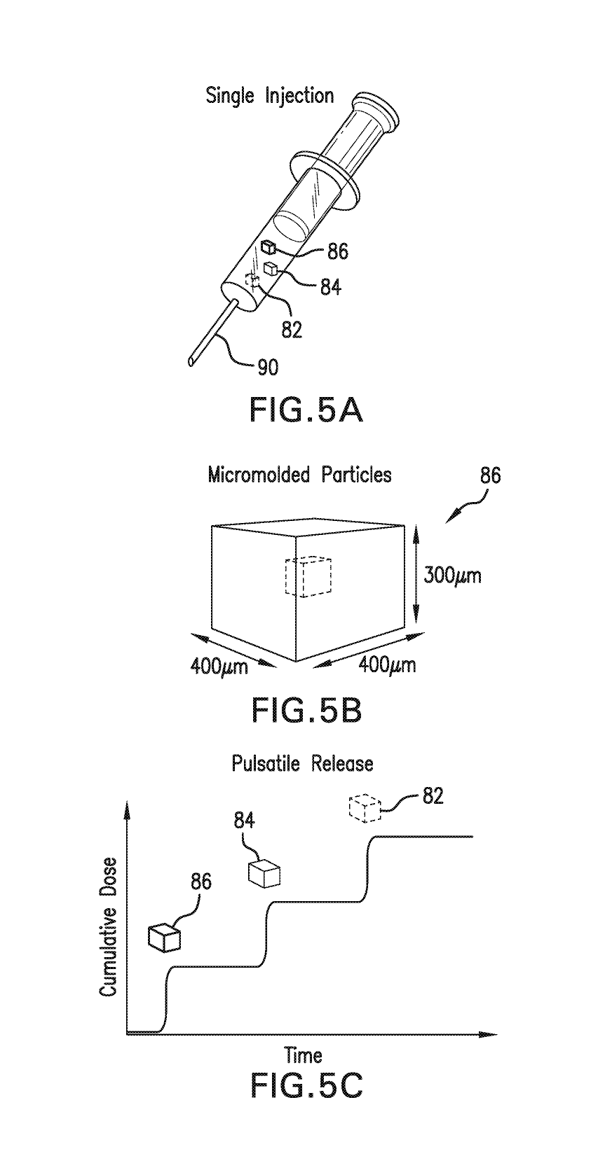

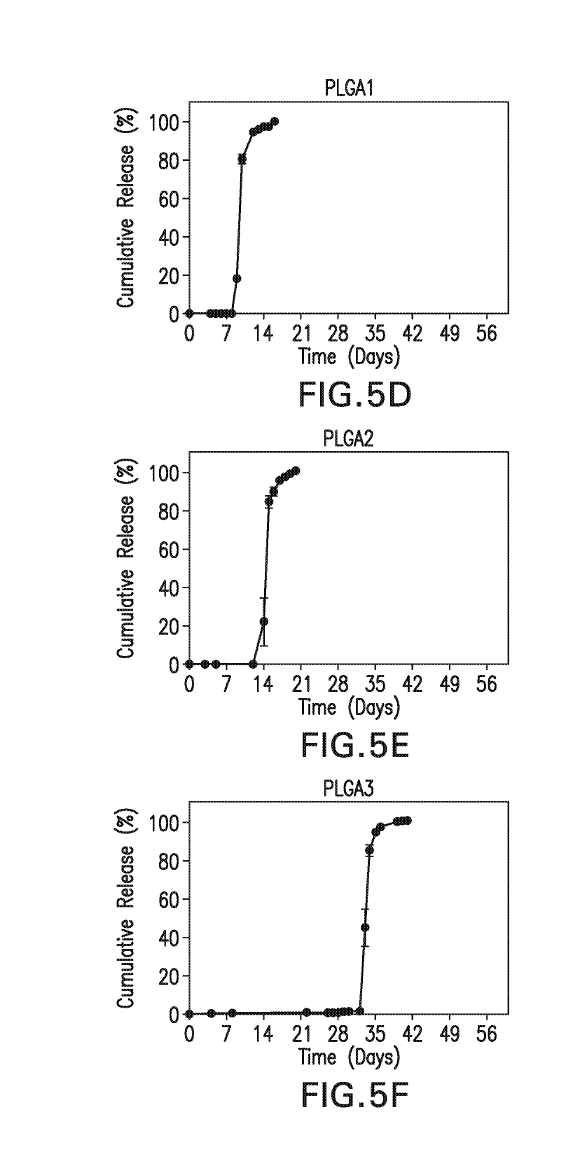

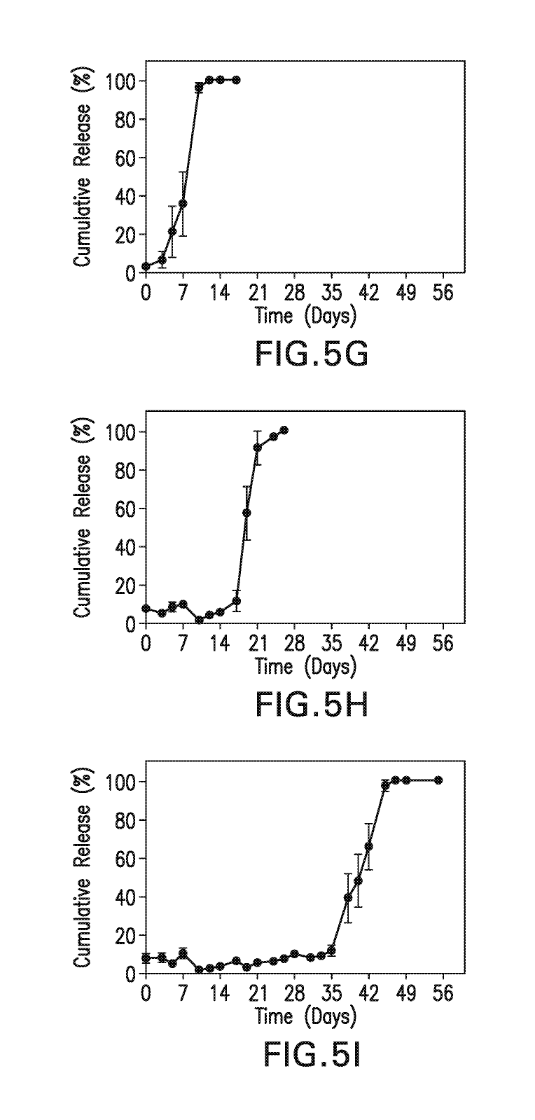

Mice were imaged using a PerkinElmer Spectrum In Vivo Imaging System (IVIS, Hopkinton, Mass.) weekly, then more frequently when approaching the projected release time. Mice were imaged 3 times per week (n=7-10, 1-2 devices/animal). At each imaging session, mice were anesthetized using continuous inhalation of 3% isoflurane and placed on the heated imaging platform. Fluorescent images were then collected using 640/700 nm or 640/720 nm excitation/emission filter sets with an F-Stop setting of 2 and subject height of 1.5 cm in Living Image 4.4 software. Cumulative release was normalized to the maximum and minimum total fluorescence in the region of interest corresponding to a particular device's complete release and background signal, respectively. Because fluorescence dropped after release due to biological clearance, values after the highest signal was achieved were set to 100% in FIGS. 5G-5I.

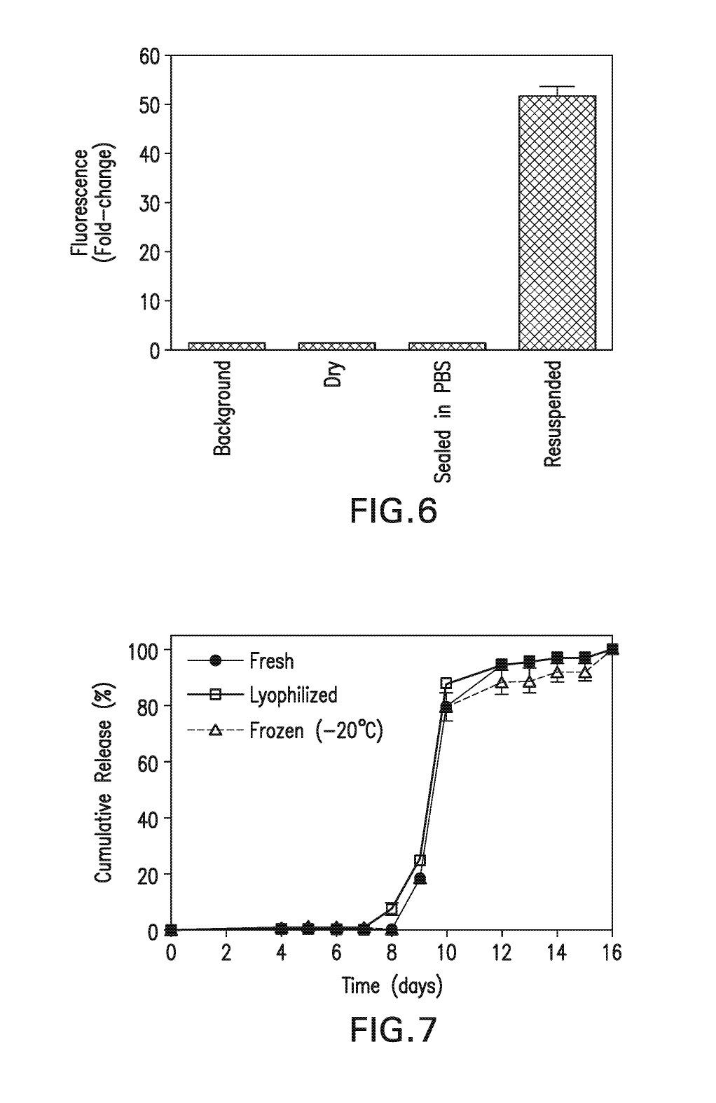

Release timing was considered to be the day on which fluorescence achieved half of its final maximum value above background. For visualization purposes, images were prepared using 3×3 smoothing, a binning setting of 4, and reported as radiant efficiency ([p/sec/cm²/sr]/[μW/cm²]). Prior to release, device-associated fluorescence was on the order of background autofluorescence, likely due to self-quenching and/or desiccation; however, the signal increased substantially upon release and spreading as depicted in FIG. 6.

To determine the effect of device number and polymer composition on release kinetics, this study was repeated with a co-injection of multiple device types. In order to allow for the differentiation of each device population, three different fluorophores were used. A mixture of 25 PLGA1 devices containing 10 μg of Alexa Fluor 594-labeled 10 kD dextran, 25 PLGA2 devices containing 10 μg of Alexa Fluor 647-labeled 10 kD dextran, and 25 PLGA4 devices containing 10 μg of Alexa Fluor 680-labeled 10 kD dextran were co-injected with an 18 G Monoject filter needle in approximately 300 μl of 15 mg/ml of methyl cellulose used as a viscosity enhancer. Mice were imaged regularly at 570/620 nm, 640/680 nm, and 675/780 nm excitation/emission, which were found to be the most effective filter sets that provided a high signal while minimizing overlap from adjacent dyes. Release timing was reported as the day between imaging sessions at which the fluorescence increased the most in the corresponding filter set.

Results

Unlike emulsion-based processes (17), loading and release kinetics of the microparticles can be decoupled. These devices can be used for controlled release of a drug after an appropriate stimulus.

The release kinetics of a model antigen from PLGA microdevices was studied. In this approach, multiple devices with different compositions could be co-injected at the time of initial immunization, degrade over time, and release antigen in discrete pulses at time points that match typical vaccination schedules (FIGS. 5A-5I).

Microdevices were fabricated using three PLGA polymers with varying properties (Table 2) and filled with fluorescently labeled dextran to observe release kinetics. Devices composed of PLGA1 released in vitro at 10±0 days, PLGA2 at 15±0, and PLGA3 at 34±1 days (FIGS. 5D-5F). No measurable leakage was observed prior to release, indicating that this platform releases its contents as a sharp pulse after degradation of the polymer barrier. A similar trend was observed when devices were subcutaneously injected into mice, as PLGA1, PLGA2, and PLGA3 devices released after 9±2, 20±1, and 41±3 days in vivo, respectively (FIGS. 5G-5I), as indicated by an approximately 50-fold increase in fluorescence upon release (FIG. 6). Images of in vivo release kinetic were taken from mice imaged using an In Vivo Imaging System following injection of a single SEAL-fabricated PLGA device (PLGA1, PLGA2, or PLGA3) containing fluorescently-labeled dextran (images not shown).

Devices could also be lyophilized or frozen at −20° C. without altering release kinetics (FIG. 7). These results are especially exciting because they enable the production of various injectable microdevices that release their payloads in distinct, delayed bursts without prior leakage. Although a number of groups have created layered microdevices using microfluidic and other approaches, these methods produce devices with continuous release, whereas the microdevices described here show rapid drug release after a material-dependent delay.

TABLE 2
Melting temperatures of polymers used with SEAL.

| Polymer | | MW | | | | GTT |
|---|---|---|---|---|---|---|
| Name | Source Material | (kD) | LA:GA | End Group | PD | (° C.) |
| PLGA1 | Evonik Resomer | 19 | 50:50 | Carboxylic | 1.79 | 42 |
| | 502H | | | Acid | | |
| PLGA2 | PolySciTech | 53 | 50:50 | Carboxylic | 1.98 | 43 |
| | AP045 | | | Acid | | |
| PLGA3 | Evonik Resomer | 75 | 50:50 | Ester | 1.85 | 48 |
| | 505 | | | | | |
| PLGA4 | Evonik Resomer | 214 | 85:15 | Carboxylic | 1.76 | 53 |
| | 858S | | | Acid | | |

MW—molecular weight,
LA—lactic acid;
GA—glycolic acid;
GTT—glass transition temperature,
PD—polydispersity

### Example 4. The Microdevices Preserve the Immunogenicity of a Thermolabile Vaccine IPV

Materials and Methods

The inactivated polio vaccine (IPV) was used as a therapeutically relevant model for sensitive biomacromolecules since it is one of the least thermostable vaccines in widespread use. IPV is rendered ineffective by heat, solvent, or other environmental factors. IPV provided by the Statens Serum Institut (Copenhagen, Denmark) at approximately 10× clinical concentration was further concentrated using Amicon Ultra-0.5 ml Centrifugal Filters with a 100 kD cutoff (EMD Millipore, Billerica, Mass.). After centrifuging the sample for 10 min at 14,000 RCF, filters were inverted and centrifuged again for 2 min at 1,000 RCF to collect the concentrated stock. This stock was then desalted to remove formulation excipients using 2 ml ZEBA Spin Desalting Columns with a 40 kD cutoff (Thermo Fisher Scientific, Waltham, Mass.). Sample was loaded into the water-equilibrated desalting columns and centrifuged for 3 min at 1,000 RCF. This process was repeated a second time to further purify the stock. The concentrated, desalted stock was then combined 1:1 with a solution of 20% (w/v) sucrose, 17% (w/v) monosodium glutamate, and 17% (w/v) MgCl₂ in water. This solution was loaded into core-shell bases using the BioJet Ultra dispensing machine in two cycles, each consisting of ten 150 pl drops for a total volume of approximately 3 nl. Devices were then sealed with an offset cap using the aforementioned sealing process at 40° C. for 3 min. Devices that were filled and capped or filled without capping were then placed in antigen dilution buffer (1% bovine serum albumin, 1% Triton X-100, 0.001% phenol red in PBS) and vortexed to recover the IPV. Samples were then diluted and analyzed using a D-antigen-specific IPV enzyme-linked immunosorbent assay (Statens Serum Institut).

Results

The compatibility of the SEAL process with biologics, including the trivalent inactivated polio vaccine (IPV), one of the most thermolabile vaccines in use today, and ovalbumin (OVA), a commonly studied model antigen, was examined.

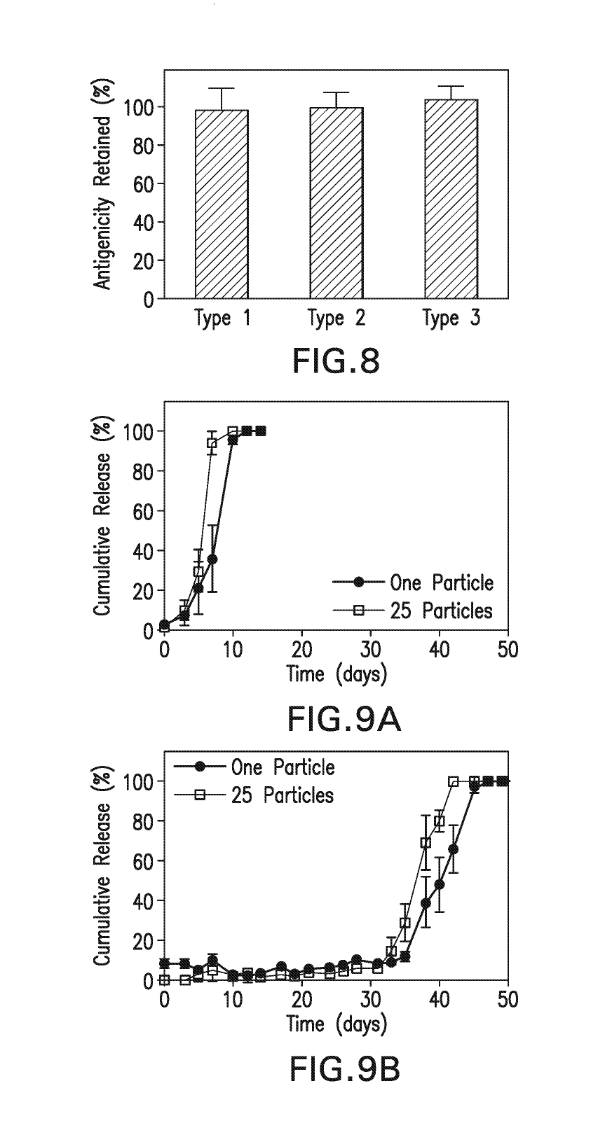

A formulation consisting of IPV, sucrose, monosodium glutamate, and magnesium chloride was dispensed into each PLGA1 device core and dried spontaneously within seconds after filling. Devices were then exposed to the sealing process to determine if brief heating (approximately 60 sec at 42° C.) had any effect on IPV stability. IPV D-antigenicity, a surrogate for the seroprotection-conferring antigen conformation used to evaluate clinical formulations, remained statistically similar before and after the sealing process, suggesting that the sealing step is relatively harmless to the encapsulated biologic (FIG. 8). Type 1 IPV retained 98.1±11.4% of D-antigenicity after filling, Type 2 IPV retained 99.6±7.8%, and Type 3 IPV retained 103.8±11.9% relative to filled, unsealed devices. Overall, the stability of the biologic during sealing in PLGA1 was much higher than what is typically reported for microsphere encapsulation using an emulsion based process, likely due to the elimination of organic solvents, emulsification stressors, and washing, which can lead to significant losses. After encapsulating and sealing IPV in PLGA microdevices, additional excipients may be needed to stabilize IPV against thermal and acidic pH stressors it will encounter during long term storage in the body. To achieve very long time points with this system, PLGA with different end groups, copolymer ratios, and/or molecular weights may be used. Maintaining a low sealing temperature to minimize stress on the antigen can be achieved by using an ester end modified PLGA cap, which increases hydrophobicity and thus delays the onset of release.

### Example 5. Microdevices as Single Injection Vaccines Require Lower Antigen Loading, and Show Adjuvant Effects

Materials and Methods

SKH1-Elite mice were injected at two sites in the rear flank with a mixture of 25 PLGA1 devices containing 10 μg of endotoxin-free OVA and 25 PLGA3 devices containing 10 μg of endotoxin-free OVA (20 m total) via an 18G filter needle in approximately 300 μl of 15 mg/ml methyl cellulose. Based on in vivo fluorescent dextran release experiments in which this quantity of mixed-population devices was injected, PLGA1 and PLGA3 devices were expected to release the largest amount of antigen 6 and 36 days after injection, respectively (FIG. 8); therefore, bolus controls were administered at these time points. Control mice received either two dose-matched injections of OVA at 6 and 36 days (20 μg total), two double-dose injections of OVA at 6 and 36 days (40 μg total), or an injection of empty (i.e. unfilled) devices on day 0 followed by 10 μg OVA injections at 6 and 36 days (20 μg total). For the group receiving 25 empty PLGA1 and 25 empty PLGA3 devices, these were injected on day 0 and then bolus OVA injections were administered at 6 and 36 days. The soluble OVA used in all control groups was also aliquoted by the BioJet dispensing system.

OVA-specific total IgG antibody titers were assessed using a blood serum enzyme-linked immunosorbent assay (ELISA) and reported as log 2 values. Mice were bled every two weeks from the submandibular vein. Serum was then isolated from whole blood by centrifuging samples at 2,000 RCF for 10 min at 4° C. and stored at −20° C. 50 μl of 20 μg/ml OVA in 100 mM carbonate-bicarbonate buffer (pH 9.6) was added to 96-well plates and incubated overnight at 4° C. Plates were then washed with 0.05% TWEEN® 20 in PBS and blocked for 1 hr at 37° C. with 5% non-fat dry milk resuspended in 0.05% TWEEN® 20 in PBS. Blocking buffer was then replaced with two-fold serial dilutions of serum diluted in blocking buffer and incubated for 2 hr at 37° C. The solution was then aspirated, washed, and replaced with a 1:1000 dilution of monoclonal goat anti-mouse IgG HRP-conjugated antibody in blocking buffer. After 1 hr incubation at 37° C., wells were washed again and 100 μl of OPD peroxidase substrate was added to the wells according to the manufacturer's instructions. The reaction was then stopped approximately 30 min later by adding 1 M H₂SO₄ before measuring absorbance at 490 nm using a Tecan Infinite M200 spectrophotometer. Titers are reported as the most dilute serum sample that yielded an absorbance reading ≥2-fold higher than serum from untreated mice at the same dilution. Statistical comparisons for peak titers were performed in GraphPad Prism (GraphPad Software, La Jolla, Calif.) using an ordinary one-way analysis of variance (ANOVA) with a Tukey multiple comparison test.

To assess “on-shelf” stability, PLGA1 devices were filled with 300 ng of EndoFit OVA and sealed. Devices were then either immediately incubated in PBS at 37° C. on an orbital shaker or stored desiccated at 4° C. for 30 days before incubation. After 12 days of incubation, the supernatant was collected, diluted, and assessed using a Chicken Egg Ovalbumin ELISA (Alpha Diagnostics International, San Antonio, Tex.) according to the manufacturer's instructions.

Results

OVA was used to study the microdevices as a single-injection vaccine. Mice received a single injection of 25 PLGA1 devices containing 10 μg of endotoxin-free OVA (EndoFit, Invivogen, San Diego, Calif.) and 25 PLGA3 devices containing 10 μg of endotoxin free OVA for a total of 20 μg. The corresponding bolus controls were injected to match the time at which 25 co-injected microdevices of each PLGA1 and PLGA3 released fluorescent 10 kD dextran (FIGS. 9A and 9B). Mice receiving OVA-filled core-shell devices achieved peak titers (20±1 on a log 2 scale) that were significantly higher than those in mice receiving two dose-matched boluses at 6 and 36 days (12±4, p<0.01).

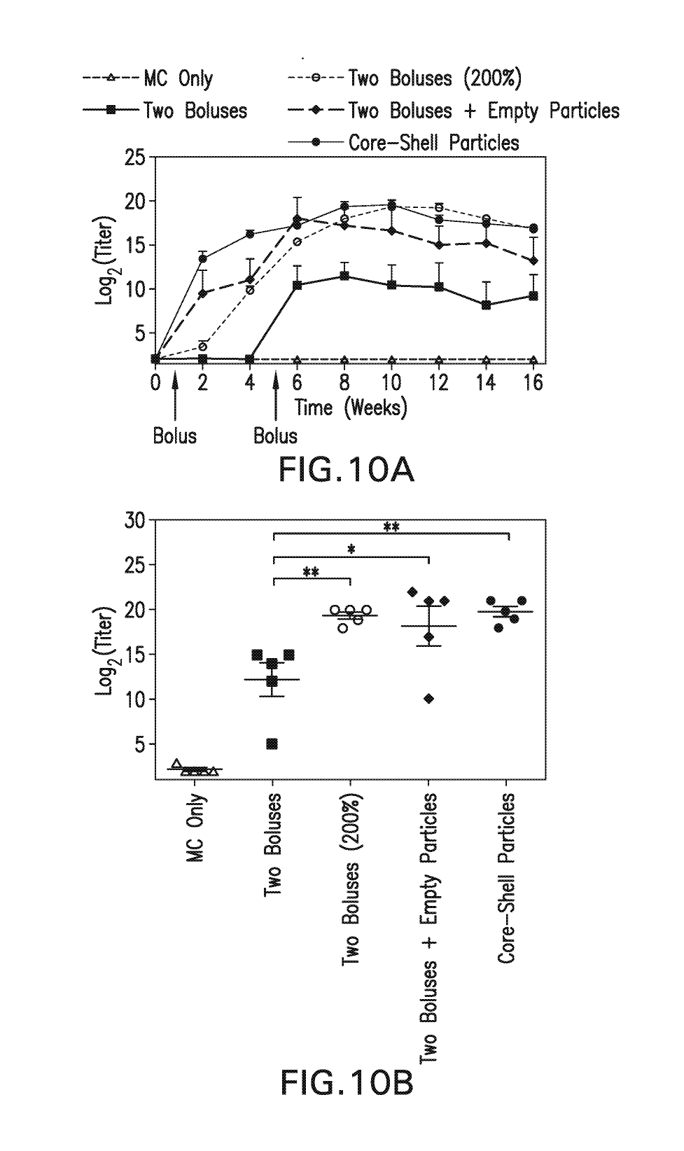

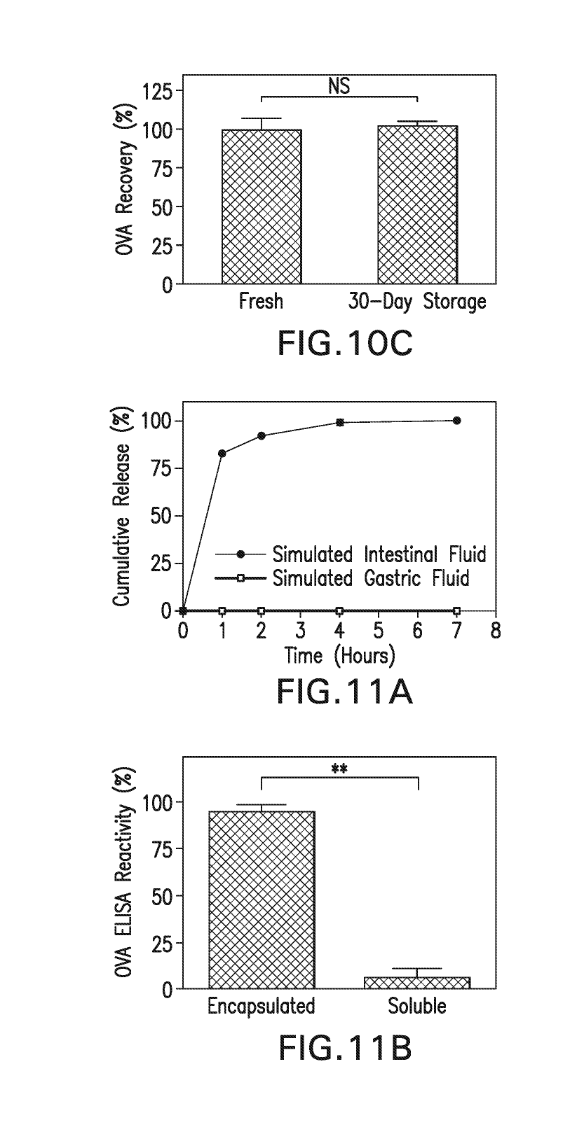

The experimental group was also statistically similar to mice receiving empty devices along with the two bolus injections (peak titer of 18±5), suggesting that the PLGA devices themselves have an adjuvant effect, consistent with what has been previously reported (26). This adjuvant effect is sufficient to achieve approximately two-fold dose sparing, since peak titers in the experimental group were statistically similar to two bolus injections containing double the antigen dose, indicating that this single-injection strategy could be used to replace multiple injections and enable the use of lower antigen doses. Longitudinal and peak antibody titers can be seen in FIGS. 10A and 10B, respectively.

Additionally, storage did not affect the release or stability of OVA, as devices stored dry for one month at 4° C. released similar amounts of ELISA-reactive OVA compared to freshly prepared devices (FIG. 10C). These experiments demonstrate that one injection of core-shell devices can induce a long term antibody response, out-perform multiple time-matched injections, and achieve two-fold dose-sparing.

### Example 6. Microdevices as Microscale Environmental pH Sensors In Vitro and In Vivo

Materials and Methods

Films of FS 30 D were created by mixing 30 g of FS 30 D aqueous dispersion (30% solids) and 1 g of triethyl citrate for 45 min at 300 RPM at room temperature. This mixture was cast on a 150 mm polystyrene petri dish and dried at 80° C. overnight. FS 30 D films were then removed and molded into device caps and bases in a 120° C. oven for 2 hr. Devices were filled with Alexa Fluor 488-labeled 10 kD dextran were incubated in 1 ml of either simulated gastric fluid (SGF, pH 1.2) or simulated intestinal fluid (SIF, pH 7.5), and placed in an Eppendorf thermomixer at 37° C. and 1000 RPM. Release was measured at 1, 2, 4, and 7 hr by analyzing the fluorescence of the supernatant as described above.

To test the ability of FS 30 D devices to protect OVA from degradation at low pH in vitro, devices filled with OVA were suspended with 1 ml SGF, and rotated (24 RPM) at 37° C. for 18 hr and compared to uncapped OVA devices that allowed for its rapid dissolution in SGF. After 18 hr, devices were transferred to 1 ml of SIF and rotated (24 RPM) for an additional 2 hr at 37° C. to release the encapsulated OVA. All samples were neutralized in SIF, diluted in sample diluent, and run on the Chicken Egg Ovalbumin ELISA according the manufacturer's instructions. Experimental samples were compared to a control group of filled devices that were only incubated in SIF for 2 hr.

In vivo release experiments were performed in SKH1-Elite mice. FS 30 D were filled with 100 ng of Alexa Fluor 750 NHS Ester (Thermo Fisher Scientific), which was chosen due to its high wavelength (e.g. low autofluorescence and tissue absorption) and statistically similar fluorescence intensity for pH 2-8 in vitro as analyzed using one-way ANOVA (p=0.23). Devices were then aligned, sealed at 40° C., and removed from the slide. Mice then received either ten devices containing Alexa Fluor 750 or an equivalent amount of dye in 200 μl of water via gavage. Mice were imaged longitudinally using IVIS at 745/800 nm ex/em while anesthetized by continuous inhalation of 3% isoflurane to observe the location and intensity of fluorescence over time. After 7 hours, mice were euthanized using carbon dioxide inhalation and had their gastrointestinal tracts removed for end-point IVIS imaging.

Results

Overall, the ability to fabricate an internal compartment alone could enable a broad array of new applications with utility in biomedicine, sensing, actuation, and environmental monitoring. Stimuli-responsive materials could be used in environmental applications to detect harmful conditions and release a molecule that either serves as a warning signal, neutralizes the harmful agent, or protects a sensitive payload until it reaches the desired target for release.

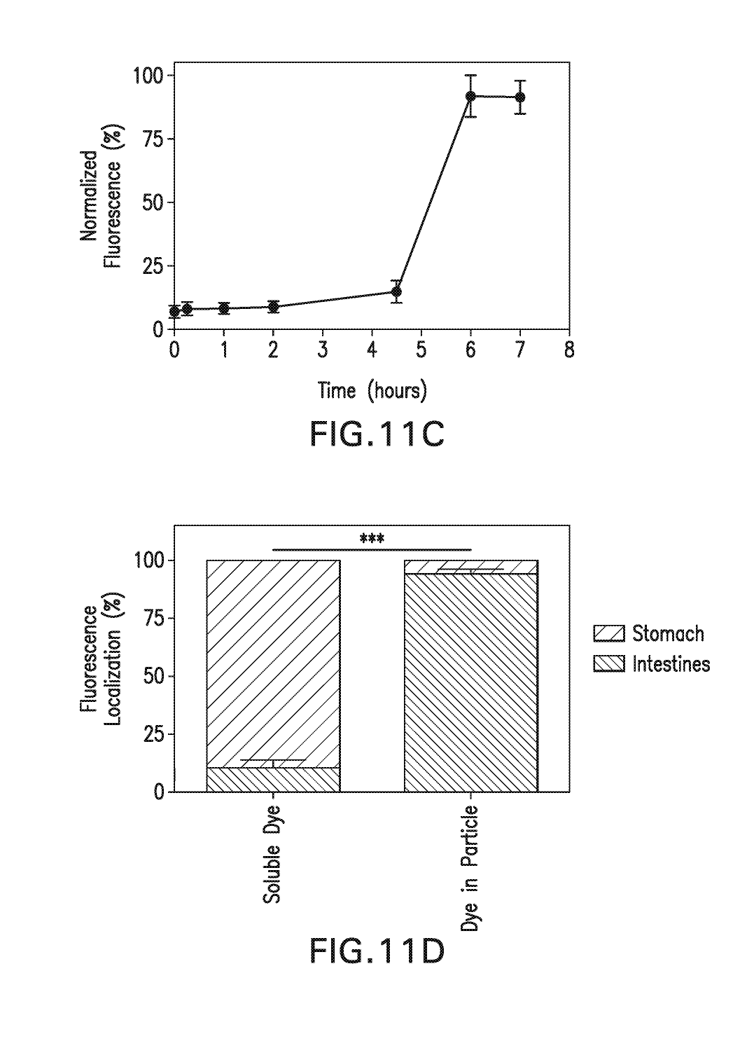

To examine this capability, a microscale pH sensor consisting of fluorescent dye encapsulated in a device composed primarily of EUDRAGIT® FS 30 D (Evonik Industries, Essen, Germany), a pH-sensitive polymer that remains solid at low pH, but rapidly dissolves at neutral pH, was generated. When subjected to a near-neutral environment (pH 7.5), these devices quickly released their contents, signaling that a change in pH had occurred (FIGS. 11A and 11B).

These devices were also able to protect a biologic from low pH insult. OVA encapsulated in FS 30 D was released at neutral pH after incubation in a pH 1.2 solution at 37° C. for 18 hr and retained 95±8% of ELISA reactivity (FIG. 11B). When fed to mice, these devices did not release their fluorescent payload in the low pH of the stomach, but only after reaching the desired target of the more neutral intestines (FIGS. 11C and 11D). Other polymers that demonstrate the opposite behavior, such as EUDRAGIT® E PO (Evonik Industries), could be used in parallel for precise regulation of environmental conditions.

### Example 7. Microdevices with Serpentine Hollow Channels

Materials and Methods

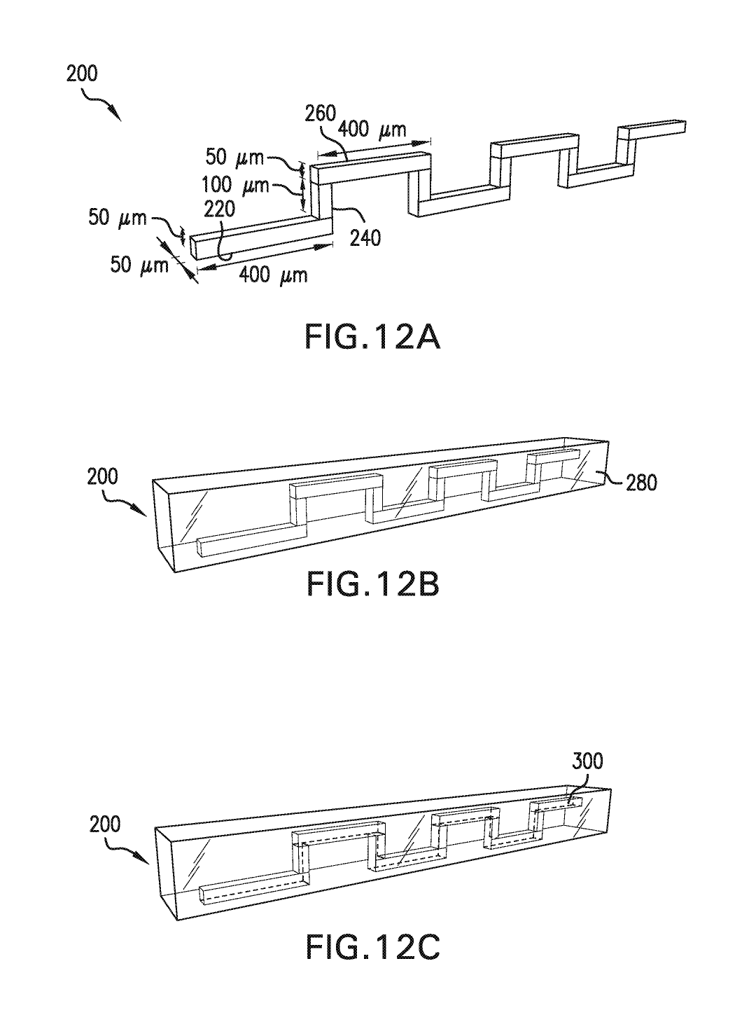

Silicon wafers 100 mm in diameter were coated with 50 μm or 100 μm of SU-8 3050 (Microchem) by spinning at a speed of 1600 or 800 RPM for 60 sec, respectively. The wafer was then baked on a hot plate at 60° C. for 5 min and 95° C. for a minimum of 15 min until excess solvent had evaporated. The wafer was then exposed using a Karl Suss MA-4 through a photomask patterned with 400×50 μm and 50×50 μm rectangles that would become the first, second, and third layer molds. The wafer was then post-baked at 60° C. for 1 min and 95° C. for 5 min. Soluble photoresist was removed from the wafer using propylene glycol monomethyl ether acetate and finished by soaking in isopropanol. These molds were treated in a chamber of tricholoro(1H,1H,2H,2H-perfluorooctyl)silane for 1 hr and used to generate PDMS inverse molds. PLGA structures were then made using these molds and sealed using the methods described above. The dimensions of the resulting three-layer microstructure based on photomask and SU-8 parameters can be seen in FIG. 12A. Sylgard 184 was then cast around the PLGA and cross-linked at 37° C. for 48 hr. After curing, PDMS was soaked in dimethylformamide for 2 hr to remove PLGA leaving behind an empty channel. The bottom channel interface and a glass slide were treated with air plasma using a Harrick Plasma Cleaner PDC-091-HP (Ithaca, N.Y.) for 1 minute on high power at 400 mTorr and pressed together to form a bond. Alexa Fluor 488-labeled 10 kD dextran was then suspended in water at 1 mg/ml and manually injected into a 50 μm microfluidic device using a 500 μl syringe and 30-gauge needle.

Results

The ability of SEAL to create 3D microfluidic channels was examined. A microfluidic channel with a cross-sectional area of 50×50 μm using a three-layered PLGA structure that was embedded in PDMS and removed via dissolution in organic solvent (FIGS. 12A-12C). The resulting structure was a vertically serpentine hollow channel in PDMS that was subsequently bonded to glass and subjected to flow. A solution of 1 mg/ml Alexa Fluor 488-labeled 10 kD dextran was manually injected into a 50 μm microfluidic device using a 500 μl syringe and 30-gauge needle. The solution was seen filling the channel both laterally and vertically through the continuous 3D serpentine channel.

These channels were optically transparent and smaller than the minimum reliable resolution produced by 3D printing, which has been reported to be 60×108 μm or 200 μm while avoiding the bonding issues associated with multiple PDMS layers.

Therefore, the SEAL method of assembly is capable of producing single-component 3D microfluidic devices. While the majority of microfluidics remain in 2D due to fabrication restrains, there are a couple of methods currently used to make 3D microfluidics. The first method is via bonding multiple polydimethylsiloxane (PDMS) layers, which is prone to leakage due to the unbonding of layers which leads to device failure. The second method is to 3D print a sacrificial layer, immerse it in PDMS, and dissolve out the printed layer. This process has inferior roughness and resolution to both the bonding method and our method. The SEAL method retains its high resolution while also making the device in one continuous piece of PDMS by assembling a sacrificial polymer structure that is then immersed in PDMS and dissolved out.

### Example 8. Modifications to the SEAL Method

The SEAL method may be modified with slight adjustments to prevent polymer overflow (“scum” formation) from PDMS mold.

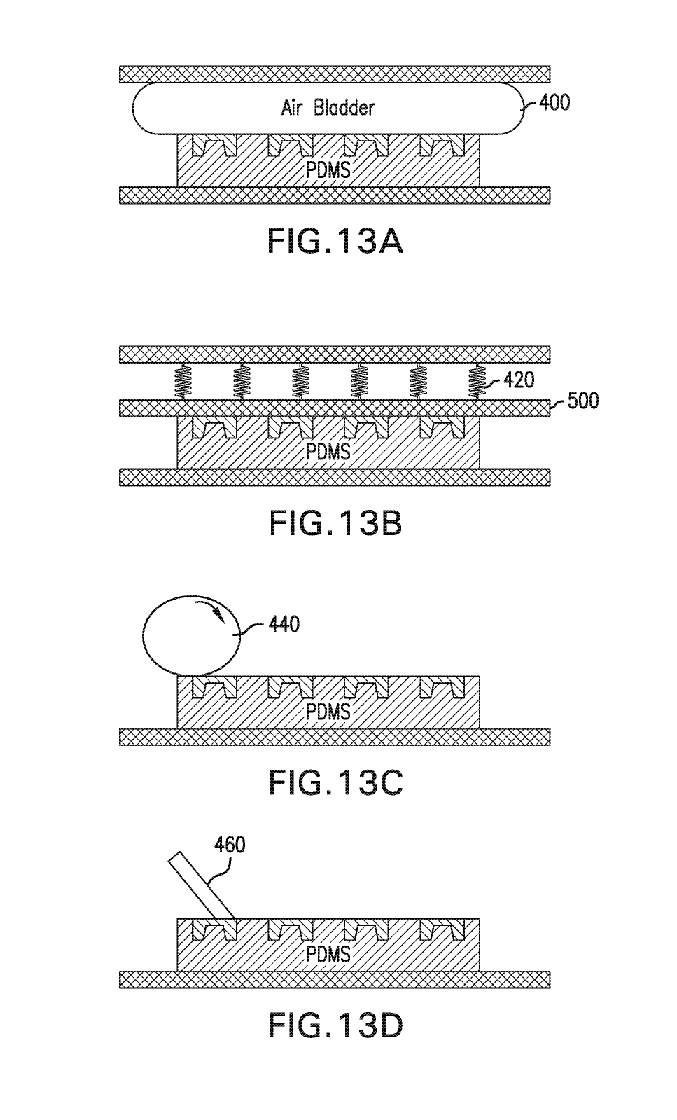

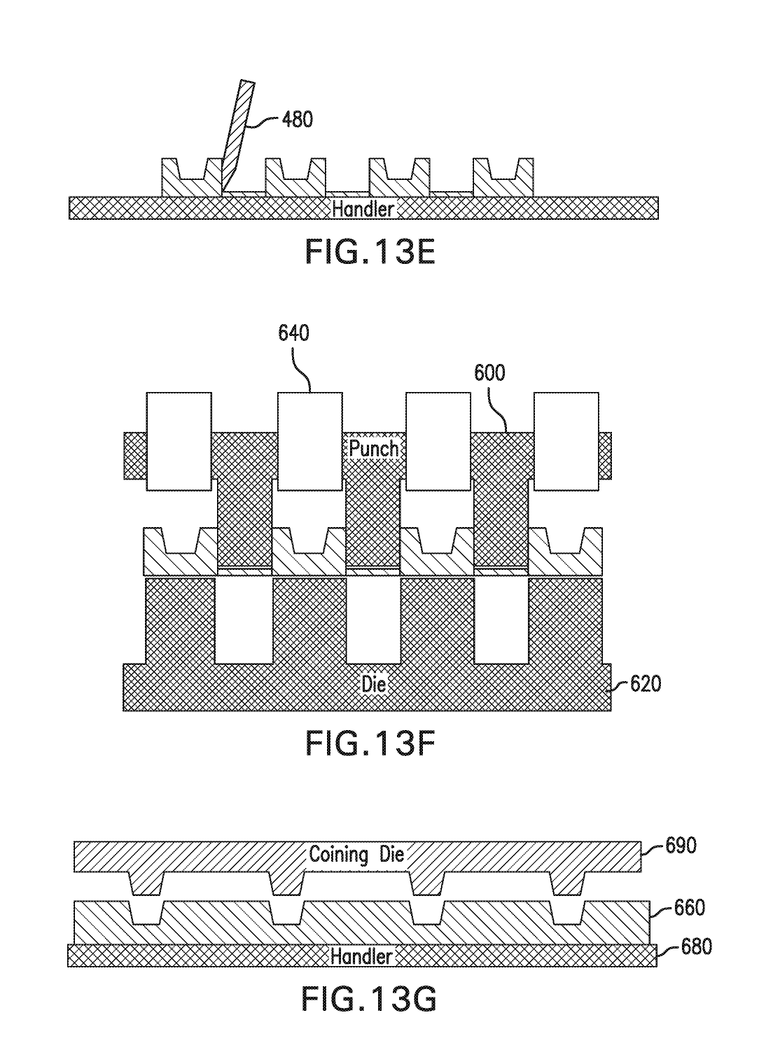

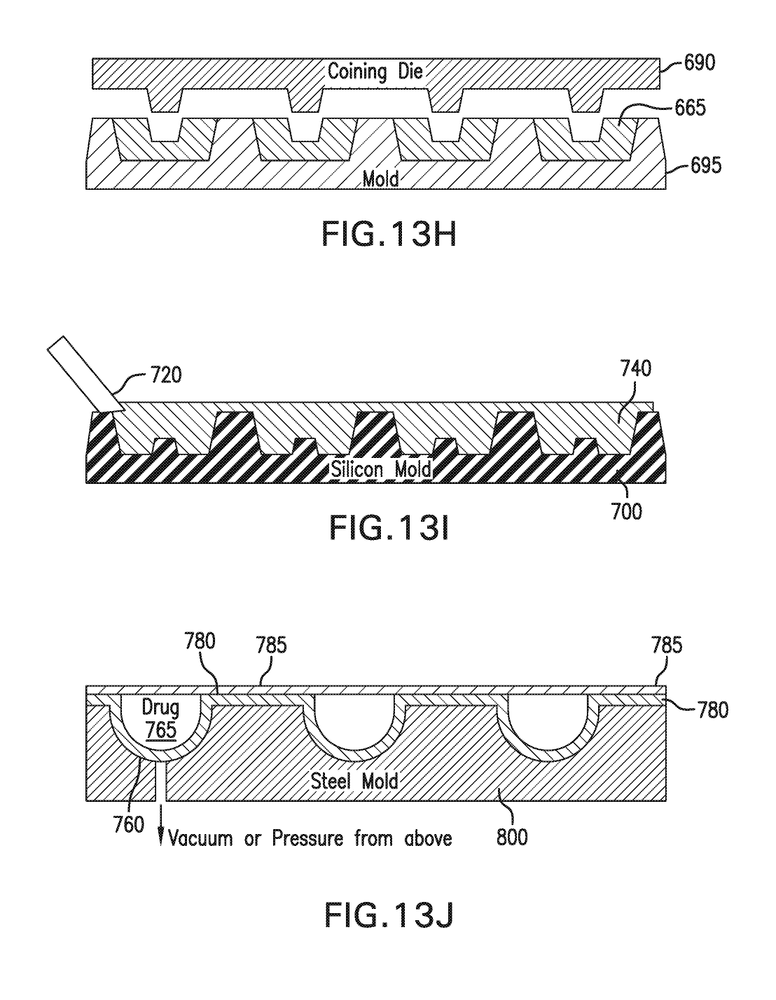

Exemplary modifications include improving polymer loading, removing the overflow, preventing overflow occurrence, and combinations thereof (FIGS. 13A-13J).

Improved polymer loading includes a uniform and controlled loading to minimize the scum layer formed during the pressing of PLGA into the silicone mold. This modification is achieved with an inclusion of an air bladder 400, a spring-loaded platen 500, or a roller 440.

For removing the overflow, a new process step is introduced to remove the scum layer more efficiently. This modification is achieved with a skiving knife 460, a laser or water jet 480, or with a punch 600 and die 620.

Preventing overflow occurrence require identifying processes that do not result in any scum generation. This modification is achieved with coining or transfer molding, or inclusion of sheet devices.

Other modifications include replacing the silicon master mold with a mold formed of other suitable materials, such as steel, or SU-8, a negative photoresist, replacing the use of microfabricated molds with a coining process to form the internal cavity of the device base, and/or assembling the top and bottom device layers via temporary solvation, polymerization, or adhesion with a biocompatible glue.

SU-8 Master Molds

The main advantages of using SU-8 as the master molds instead of etching into silicon are that it is less expensive, requires fewer instruments, faster, and leads to smoother and straighter features. This process would consist of one or more cycles of SU-8 photoresist deposition and patterned exposure followed by a development step to create microstructures on top of the substrate.

Coining

The ability to coin the device cores (i.e. stamp out the core structure) rather than creating a microfabricated mold may automate and scale up device fabrication rate.

Alternative Adhesion Methods

Using polymerization, solvation, or glue to assemble the layers of the devices may avoid damaging sensitive biologics that are unable to withstand brief heating (typically 40-60° C. depending on the shell material).

Examples of Modified Methods

FIGS. 13A-13J are diagrams showing modifications to the SEAL method.

FIG. 13A shows the use of an air bladder 400 which may eliminate the need for ensuring rigid platens are parallel.

FIG. 13B shows the use of springs 420 to allow top platen 500 to adjust and apply reasonably uniform pressure across entire surface.

FIG. 13C shows the use of a roller 440, which applies localized pressure, equally across material. The roller may be spring loaded (provides force control instead of position control, which could result in excessively high forces).

FIG. 13D shows scum layer may be removed prior to filling. The skiving knife 460 removes scum layer, but then a “handler” needs to be attached to particles to keep all devices together for filling; then the devices are singulated.

FIG. 13E shows a vision system may be combined with a computer controlled laser 480 and requires no manual labor.

FIG. 13F shows a punch 600 and die 620 modification, which can be very effective once alignment/registration is achieved. Ejection pins 640 are present on punch 600 and die 620 side (only punch side shown). With this method it may be possible to eject devices directly on to handling layer.

FIG. 13G shows a coining operation eliminates scum layer, it keeps top of devices planar, and no alignment is required for the coining operation. PLGA is formed as sheet 660 and attached to a handling wafer 680, the coining die 690 forms cavities, and singulation is required, such as with a laser, a punch and die, or other methods.

FIG. 13H shows another coining operation, which eliminates scum layer, and keeps the top of the devices planar. Alignment is required for coining operation, but singulation is not required. PLGA is dispensed into individual device cavities in a mold 695. After initial setup, coining die 690 is introduced into PLGA to form devices 665, which remain on the coining die 690. The mold 695 is removed, and a handling wafer (not shown) is introduced to keep devices together.

FIG. 13I shows a modified method with the PDMS mold replaced with a silicon mold 700. The process is simplified with no PDMS mold and no loose devices until post capping. PLGA is dispensed directly into a silicon mold 700, pressure is applied, a skiving knife 720 removes the scum layer, a handling wafer is attached to PLGA devices 740, and the PLGA devices 740 are released from the silicon mold 700. This method may require a release agent, temperature, etc.

FIG. 13J shows a simplified process (no PDMS mold, no scum layer), no loose devices are formed until post capping, with an option to form spherical microdevices 760, which may include two halves, or folding over itself. A PLGA sheet 780 is placed over a steel mold 800 with dimples, vacuum is pulled, or pressure from above is applied (sheets need to be at elevated temperature), PLGA cavity is filled with a drug 765 via BioDot, upper PLGA sheet 785 is applied with heat to seal devices 760, the devices 760 are then separated (multiple approaches), and removed from mold.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A microdevice having dimensions of less than one centimeter comprising
a micromolded fillable polymeric shell comprising a non-photoactivatable biocompatible polymer having a complex channel or core therein.

2. The microdevice of claim 1, wherein the channel or core contains agent to be released or modify a property of the microdevice.

3. The microdevice of claim 2 further comprising a cap sealing the channel or core.

4. The microdevice of claim 1 wherein the polymer comprises a biocompatible polymer is selected from the group consisting of biodegradable polymers, phase-change polymers, thermoplastic polymers, and combinations thereof.

5. The microdevice of claim 1, wherein the microdevice dimensions are between about 1 micrometer (μm) and 1000 μm, and wherein the channel or core dimensions are less than 800 μm.

6. The microdevice of claim 1, wherein the microdevice has a loading capacity between 1 percent weight/weight (% w/w) and 50% w/w.

7. The microdevice of claim 1, wherein the volume of the hollow core allows for loading the agent at between 1 percent (%) and 50% of the total volume of the microdevice.

8. The microdevice of claim 1, wherein the agent is a therapeutic, prophylactic, nutraceutical, or diagnostic or imaging agent, optionally in combination with excipient.

9. The microdevice of claim 8, wherein the agent is released from discrete regions of the microdevice with different release kinetics.

10. The microdevice of claim 8, wherein the agent is one or more antigens, wherein the antigen is released in a defined time period in an amount effective to enhance or tolerize an immune response in vivo.

11. The microdevice of claim 8, comprising excipient selected from the group consisting of sugars, salts, oils, lipids, and carbohydrates.

12. The microdevice of claim 1 comprising metal, ceramic, or other polymer property modifying agent.

13. The microdevice of claim 1 for electromechanical use comprising agent modifying material properties, electroconductivity or viscosity of the polymer or agent therein.

14. The microdevice of claim 1 comprising agents that act as sensors for environmental changes, such as pH sensors, or as sinks for reacting with, and neutralizing toxins.

15. The microdevice of claim 1 detecting and releasing agent in response to pH or enzymes indicative of infection, inflammation or cancer.

16. The microdevice of claim 1 made by a method comprising
micromolding a polymeric base having a channel or hollow core therein;
inserting agent into the channel or core;
placing a polymeric cap on the polymeric base; and
sealing the polymeric cap to the polymeric base.

17. The microdevice of claim 16, wherein the step of micromolding the polymeric base further comprises layer-by-layer sintering under microscopic alignment.

18. The microdevice of claim 16 wherein the scrum is removed from prior to placement of the polymeric cap.

19. A method of delivery of an agent to an individual comprising administering to the individual the microdevice of claim 1.

20. A method of making the microdevice of claim 1, comprising
micromolding a polymeric base having a channel or hollow core therein;
inserting agent into the channel or core;
placing a polymeric cap on the polymeric base; and
sealing the polymeric cap to the polymeric base.

21. The method of claim 20, wherein the step of micromolding the polymeric base further comprises layer-by-layer sintering under microscopic alignment.
